(19) United States
(12) Patent Application Publication
(10) Pub. No.: US 2011/0319766 A1
(43) Pub. Date: Dec. 29, 2011

(54) MEASURING DEVICE, BIOLOGICAL TESTING DEVICE, FLOW VELOCITY MEASURING METHOD, AND PRESSURE MEASURING METHOD

(75) Inventor: Jiro TSURUNO, Chino (JP)

(73) Assignee: SEIKO EPSON CORPORATION, Tokyo (JP)

(21) Appl. No.: 13/165,933

(22) Filed: Jun. 22, 2011

(30) Foreign Application Priority Data
Jun. 25, 2010 (JP) 2010-145107

(51) Int. Cl. A61B 8/06

(52) U.S. Cl. 600/454

(57) ABSTRACT

A measuring device includes an ultrasonic sensor with ultrasonic arrays each having a linear array structure in which ultrasonic elements are arranged along a linear scanning direction with the linear scanning directions of at least two of the ultrasonic arrays being different from each other, a transmission/reception control unit that controls transmission/reception of ultrasonic waves by the ultrasonic arrays, and a computation part that measures a frequency shift amount based on a reception signal from the ultrasonic arrays. The transmission/reception control unit includes a signal delay circuit that controls a transmission angle of ultrasonic waves. The computation part includes a frequency shift amount calculating part that calculates, for each of the ultrasonic arrays, a frequency shift amount based on a reception signal from each of the ultrasonic arrays, and a maximum shift amount obtaining part that obtains a maximum frequency shift amount from the calculated frequency shift amounts.

### CROSS-REFERENCE TO RELATED APPLICATIONS

This application claims priority to Japanese Patent Application No. 2010-145107 filed on Jun. 25, 2010. The entire disclosure of Japanese Patent Application No. 2010-145107 is hereby incorporated herein by reference.

### BACKGROUND

1. Technical Field

The present invention relates to a measuring device, a biological testing device, a flow velocity measuring method, and a pressure measuring method for using ultrasound to measure the state of an object to be measured.

2. Related Art

The ultrasound Doppler method is conventionally known as a method for measuring the velocity of movement of a fluid using ultrasound. This ultrasound Doppler method is a method in which ultrasound having a predetermined frequency is directed on a fluid, and the flow velocity is obtained from the amount by which the frequency of the resulting reflected wave shifts. The flow velocity can be obtained using a relationship shown in the following Equation (1), where Δf is the amount of frequency displacement, f0 is the frequency of the incident ultrasound, v is the flow velocity, γ is the angle between the direction of travel of ultrasound and the direction of fluid flow, and c is the speed of sound.

$$\begin{matrix}Equation ( 1 ) & \\ Δ f = \frac{2 {f}_{0} \cdot V \cdot cos γ}{c} & ( 1 )\end{matrix}$$

According to an ultrasound Doppler method such as that described above, the frequency shift amount cannot be measured in an instance in which angle γ is 90°, as shown in the above Equation (1). Also, the error is known to decrease as γ decreases. Therefore, in order to measure the flow velocity using the ultrasound Doppler method, it is necessary to set the angle γ between the ultrasound transmission angle and the direction of fluid flow (referred to as “ultrasound incidence angle” hereafter) to an optimum angle. Devices for this purpose have been developed (e.g., see Japanese Laid-Open Patent Application Publication No. S58-54940, Japanese Laid-Open Patent Application Publication No. 2009-89911, and Japanese Laid-Open Patent Application Publication No. 2008-220662).

According to a combined ultrasound diagnosis device described in Japanese Laid-Open Patent Application Publication No. S58-54940, an ultrasound probe comprising a plurality of piezoelectric transducers is arranged so as to form an arc surface whose side towards the sound radiating surface is convex. An ultrasonic beam is sent out to a scan region that has a radial shape radiating from the center of the arc of the probe, and that is delimited at the position of the probe. It thereby becomes possible to set a preferred angle of the ultrasonic beam in relation to the direction of blood flow (i.e., ultrasound incidence angle).

Also, in Japanese Laid-Open Patent Application Publication No. 2009-89911, there is disclosed a method in which a device comprising two minor-axis ultrasonic array probes and a major-axis ultrasonic array probe is used, the two minor-axis ultrasonic array probes being arranged in parallel, and the major-axis ultrasonic array probe being disposed orthogonal to the minor-axis ultrasonic array probes. Positional alignment is performed so that the respective distance from each of the two minor-axis ultrasonic array probes to the center of the blood vessel is equal.

Also, according to Japanese Laid-Open Patent Application Publication No. 2008-220662, a probe is used to perform a three-dimensional volume scan, and the resulting volume data is obtained. Then, the position of the sound rays used in the Doppler measurement, the sampling marker position, the clipping range angle, and the clipping range are set based on this volume data, and a clipping image is generated and displayed on a monitor.

### SUMMARY

In a device such as that described in above-mentioned Japanese Laid-Open Patent Application Publication No. S58-54940, the user must perform work to make the direction along which the probe is arranged and the axial direction of the blood vessel parallel to each other. Also, in the method described in Japanese Laid-Open Patent Application Publication No. 2009-89911, the user must perform work to align the axial direction of the blood vessel and the direction of the major axis. Therefore, according to those publications, a problem is presented in that a user with no professional knowledge will be unable to readily set the ultrasound incidence angle γ to an optimum level, and will be unable to obtain an optimum frequency shift amount to measure the blood flow to a high degree of accuracy.

In the device described in Japanese Laid-Open Patent Application Publication No. 2008-220662, a three-dimensional image is used to make it easier to visually identify the portion at which the blood flow is to be measured. However, a complex configuration becomes necessary to perform image processing. A problem is also presented in that an auxiliary device becomes necessary in order for the operator to visually identify the position of the blood vessel or another element and clearly specify the measurement position on a diagnosis device, resulting in increasing cost and reduced portability.

With the problems described above in view, it is an object of the present invention to provide a measuring device, a biological testing device, a flow velocity measuring method, and a pressure measuring method using which it is possible to readily obtain an optimum frequency shift amount for measuring blood flow.

A measuring device according to one aspect of the present invention includes an ultrasonic sensor, a transmission/reception control unit, and a computation part. The ultrasonic sensor has a substrate with a plurality of ultrasonic arrays each having a linear array structure in which a plurality of ultrasonic elements are arranged along a linear scanning direction so that linear scanning directions along which the ultrasonic elements are arranged are different from each other at least for two of the ultrasonic arrays that are arranged on the substrate. The transmission/reception control unit is configured to control the ultrasonic arrays to transmit/receive ultrasonic waves. The computation part is configured to measure a frequency shift amount based on ultrasonic waves received by the ultrasonic arrays.

In addition to an aspect in which the ultrasonic sensor, the transmission/reception control unit, and the computation part are arranged in one device, the measuring device according to the aspect of the present invention also includes, e.g., a system in which a control device, which is provided with the transmission/reception control unit and the computation part, and the ultrasonic sensor are configured separately and connected so as to make communication possible; as well as other aspects.

Also, in addition to an aspect in which a plurality of ultrasonic elements capable of performing both the transmission and reception of ultrasonic waves are arranged, the ultrasonic arrays also include, e.g., an aspect in which both ultrasonic elements for ultrasound transmission and ultrasonic elements for ultrasound reception are provided in a single ultrasonic array, or an aspect in which both ultrasonic arrays for ultrasound transmission and ultrasonic arrays for ultrasound reception are provided in an ultrasonic sensor, as well as other aspects.

In the measuring device according to the aspect described above, the transmission/reception control unit performs a control procedure for causing ultrasonic waves to be transmitted/received by each of the ultrasonic elements of the ultrasonic arrays. The frequency of the transmitted ultrasonic waves shifts upon being reflected by a measured fluid. When the ultrasonic waves are received by an ultrasonic array, the computation part measures the frequency shift amount from a resulting reception signal.

At this time, according to the aspect of the present invention, the plurality of the ultrasonic arrays are arranged on the substrate so that the linear scanning directions are different from each other. Accordingly, there is no need to perform position alignment of the ultrasonic sensor with respect to, e.g., the position of the blood vessel in order to obtain the frequency shift amount. Therefore, even for a user without specialist knowledge, it is possible to obtain the frequency shift amount merely by wearing the ultrasonic sensor at the measurement position, without any particular consideration for, e.g., the mounting angle of the ultrasonic sensor.

Also, in an aspect in which a plurality of ultrasonic arrays having a linear array structure are arranged on the substrate, costs are reduced, and wires connected to the ultrasonic elements of each of the ultrasonic arrays can be more readily arranged, compared to an aspect in which ultrasonic arrays having a two-dimensional array structure are arranged in a spread-out arrangement.

In the measuring device as described above, the transmission/reception control unit preferably includes a delay control unit configured to control a transmission angle of ultrasonic waves transmitted from the ultrasonic arrays.

In the measuring device according to the aspect described above, the delay control unit delays the transmission timing of ultrasonic waves transmitted from each of the ultrasonic elements of each of the ultrasonic arrays and thereby varies the ultrasound transmission angle. Therefore, when a straight line that joins each of the ultrasonic elements of an ultrasonic array in the same direction as the linear scanning direction is the linear scan line, it becomes possible to cause the ultrasonic array to vary the transmission angle of ultrasonic waves within a plane that passes through the linear scan line and that is orthogonal to the substrate surface.

Also, it is known that generally, in an instance in which the flow velocity of the measured fluid is measured based on the above Equation (1), the error when calculating the flow velocity decreases with decreasing ultrasound incidence angle (i.e., the angle between the direction of ultrasound transmission and the direction of the flow path of the measured fluid). In other words, when the flow velocity is calculated based on Equation (1), the error in the calculated flow velocity decreases, and it becomes possible to calculate the flow velocity to a higher degree of accuracy, with increasing frequency shift amount. Therefore, in the aspect of the present invention, the delay control unit varies the transmission angle of the ultrasound, thereby making it possible to select a larger frequency shift amount in the computation part, and to obtain a more suitable frequency shift amount.

In the measuring device as described above, the computation part preferably includes a frequency shift amount calculating part configured to calculate, for each of the ultrasonic arrays, the frequency shift amount based on a reception signal outputted from each of the ultrasonic arrays, the frequency shift amount being a difference between a frequency of transmitted ultrasonic waves and a frequency of received ultrasonic waves, and a maximum shift amount obtaining part configured to obtain a maximum frequency shift amount, which is the largest of the frequency shift amounts calculated for each of the ultrasonic arrays by the frequency shift amount calculating part.

In the above described aspect, the plurality of ultrasonic arrays are arranged on the substrate so that the linear scanning directions are different from each other. The frequency shift amount calculating part of the computation part obtains, from each of the ultrasonic arrays, the frequency shift amount with respect to the respectively different ultrasound incidence angle γ, and the maximum shift amount obtaining part of the computation part obtains the maximum frequency shift amount, which is the largest of these frequency shift amounts.

As described above, in an instance in which the flow velocity is calculated based on Equation (1), the error in the calculated flow velocity decreases, and it becomes possible to calculate the flow velocity to a higher degree of accuracy, with increasing frequency shift amount. Therefore, in the aspect of the present invention, the maximum frequency shift amount is obtained as described above, thereby making it possible to obtain a frequency shift amount with respect to an optimum ultrasound incidence angle at which the error when measuring the blood flow is smaller.

In the measuring device as described above, the transmission/reception control unit is preferably configured to control the ultrasonic sensor to transmit/receive ultrasonic waves at a plurality of timings. The frequency shift amount calculating part is preferably configured to calculate the frequency shift amount at each of the timings, based on the reception signal outputted from one of the ultrasonic arrays corresponding to the maximum frequency shift amount obtained in the maximum shift amount obtaining part at a previous timing.

According to the aspect described above, the transmission/reception control unit causes the ultrasonic sensor to perform an operation of transmitting/receiving ultrasonic waves at a plurality of timings. With regards to the plurality of timings for receiving ultrasonic waves, transmission/reception of ultrasonic waves may be, e.g., performed at a periodic timing, performed continuously, or performed at a previously set time or at a time set by the user. When the maximum frequency shift amount is obtained by the maximum shift amount obtaining part, the frequency shift amount calculating part calculates the frequency shift amount based on the reception signal outputted from the ultrasonic array that outputted the reception signal for which the maximum frequency shift amount was calculated, where the calculated frequency shift amount is considered the maximum frequency shift amount for the corresponding timing. In a measuring device of such description, it is possible to obtain, e.g., the variation over time of the measured fluid. In particular, in an instance in which the measured fluid is blood, and the state of blood flowing through a blood vessel in the body is measured, it is possible to measure, e.g., the state of blood flow (e.g., blood flow, blood pressure, pulse, or a similar variable) over 24 hours, in a detailed manner. Specifically, the state of blood flow in a body may vary during everyday life; even if no abnormality is present in the state of blood flow at one time, an abnormality may be present at another time. Therefore, the measuring device according to the aspect of the present invention can be used to measure the state of blood flow at a plurality of timings over a long period, and thereby provide support for early discovery in an instance in which an abnormality is present in the state of blood flow.

Also, in the measuring device as described above, the transmission/reception control unit is preferably configured to control the ultrasonic sensor to transmit/receive ultrasonic waves at a plurality of timings, the frequency shift amount calculating part is preferably configured to calculate the frequency shift amount at each of the timings based on the reception signal outputted from each of the ultrasonic arrays, and the maximum shift amount obtaining part is preferably configured to obtain the maximum frequency shift amount from the frequency shift amount each time a calculation is made by the frequency shift amount calculating part.

In this instance, as with the aspect described in the foregoing, it is possible to obtain the variation over time of the measured fluid, and e.g., in an instance in which the blood flow in a body is measured, it is possible to measure the state of blood flow over a long period, and thereby support, in a satisfactory manner, the user in maintaining health. In addition, according to the aspect described above, the frequency shift amount calculating part calculates the frequency shift amount in each of the ultrasonic arrays at every ultrasound transmission/reception timing, and the maximum shift amount obtaining part obtains the maximum frequency shift amount from the frequency shift amount for each of the ultrasonic arrays each time these frequency shift amounts are calculated. Therefore, in an instance in which the flow path of the measured fluid changes, e.g., in an instance in which movement of the body changes the position of the blood vessel in the body, it is possible to obtain the optimum maximum frequency shift amount at all times. Therefore, in an instance in which the flow velocity of the measured fluid is measured using the maximum frequency shift amount of such description, it is possible to measure the flow velocity to a high degree of accuracy with minimal error.

Also, in the measuring device as described above, the transmission/reception control unit is preferably configured to control the ultrasonic sensor to periodically transmit/receive ultrasonic waves.

It is difficult to calculate the frequency component of ultrasonic waves, based on a reception signal, based on ultrasonic waves received at an arbitrary timing. The accuracy of calculating the frequency shift amount also deteriorates. In contrast, in an instance in which transmission/reception of ultrasonic waves is performed periodically and the frequency shift amount is calculated based on a reception signal obtained periodically as in the aspect of the present invention, the frequency shift amount calculating part is able to calculate the frequency shift amount using an arithmetic algorithm that uses FFT (i.e., fast Fourier transform), and it is possible to determine the frequency shift amount to a high degree of calculation accuracy and at high speed.

In the measuring device as described above, the computation part preferably includes a reception period measuring part configured to measure a reception period between transmission of ultrasonic waves and reception of reflected ultrasonic waves in the one of the ultrasonic arrays corresponding to the maximum frequency shift amount or another one of the ultrasonic arrays, a reflection position calculating part configured to calculate a reflection position at which ultrasonic waves are reflected, based on data relating to a position of the ultrasonic array, the reception period, and a transmission angle at which ultrasonic waves are transmitted from the ultrasonic array, and a movement direction measuring part configured to determine a direction of movement of a measured fluid from the reflection position calculated by the reflection position calculating part.

According to the aspect described above, the reception period measuring part measures the reception period between transmission and reception of ultrasonic waves. The reflection position calculating part calculates the direction of movement of the measured fluid based on this reception period, the angle at which each of the ultrasonic waves is beamed, and position data showing a position at which each of the ultrasonic arrays is arranged on the ultrasonic sensor. Specifically, in an instance in which the scan range of the ultrasonic sensor is sufficiently small, the direction of a straight line linking two reflection positions detected by two ultrasonic arrays can be regarded as the direction of movement of the measured fluid. The direction of movement of the measured fluid coincides with the direction in which a tube, through which the measured fluid flows, is arranged. Therefore, the movement direction measuring part as described above can be used to measure the position of the tube.

Also, in the measuring device as described above, the ultrasonic sensor preferably includes a plurality of position-measuring ultrasonic arrays configured and arranged to measure a position of a tube through which a measured fluid flows, and the computation part preferably includes a movement direction calculating part configured to calculate a direction of movement of the measured fluid in the tube based on a reception signal outputted from the position-measuring ultrasonic arrays.

In this instance, even though the position-measuring ultrasonic arrays are separately required, using position-measuring ultrasonic arrays of such description makes it possible to output a frequency that is exclusive for position measurement from the position-measuring ultrasonic arrays, and to measure the direction of movement of the measured fluid, i.e., the position of installation of the tube through which the measured fluid flows.

The measuring device as described above preferably further includes a flow velocity calculating part configured to calculate a flow velocity of the measured fluid based on a direction of movement of the measured fluid, the maximum frequency shift amount, and a frequency of ultrasonic waves transmitted from the ultrasonic array.

According to the aspect described above, the flow velocity calculating part calculates the flow velocity of the measured fluid based on the above Equation (1), using the direction of movement of the measured fluid (i.e., installation position of the tube), the maximum frequency shift amount, and the frequency of the transmitted ultrasonic waves.

As with the aspect described in the foregoing, the maximum shift amount obtaining part obtains the maximum frequency shift amount from the frequency shift amount calculated based on the reception signal outputted from each of the ultrasonic arrays. Therefore, the flow velocity calculating part is able to use a maximum frequency shift amount corresponding to an optimum ultrasound incidence angle for calculating the flow velocity to a high degree of accuracy. Also, it is possible to establish the position of the ultrasonic array that outputted the reception signal used to calculate the maximum frequency shift amount, and it is therefore possible to calculate an accurate ultrasound incidence angle based on the position data with regards to this ultrasonic array and the direction of movement of the measured fluid. Therefore, the flow velocity calculating part is able to determine the flow velocity based on an optimum maximum frequency shift amount and an optimum ultrasound incidence angle corresponding to this maximum frequency shift amount. In other words, according to the measuring device of the aspect of the present invention, it is possible to readily measure the flow velocity of the measured fluid to a high degree of accuracy with minimal error.

The measuring device as described above preferably further includes a diameter obtaining part configured to obtain a diameter of a flow path in which the measured fluid flows, and a pressure measuring part configured to measure a pressure of the measured fluid based on the diameter of the flow path and the flow velocity of the measured fluid.

According to the aspect described above, the diameter obtaining part obtains the diameter of the flow path of the measured fluid, i.e., the tube diameter, and the pressure measuring part computationally determines the fluid pressure of the measured fluid based on the flow velocity of the measured fluid calculated as described above and the diameter of the flow path. In this instance, the pressure is calculated based on the flow velocity of the measured fluid whose error is minimal as described above, and it is therefore possible to calculate the pressure to a high degree of accuracy. Also, as described above, it is possible to readily determine the flow velocity computationally using a simple configuration, and it is therefore possible to readily determine the pressure computationally using a simple configuration.

In the measuring device as described above, the ultrasonic sensor preferably includes a plurality of diameter-measuring ultrasonic arrays configured and arranged to measure the diameter of the flow path, and the diameter obtaining part is configured to calculate the diameter of the flow path based on a reception signal outputted from the diameter-measuring ultrasonic arrays.

In this aspect, it is possible to measure the tube diameter based on the time taken until the ultrasonic waves reflected at the tube wall nearer the ultrasonic arrays to be received by the ultrasonic arrays and the time taken for the ultrasonic waves reflected at the tube wall further from the ultrasonic arrays to be received by the ultrasonic arrays. However, in an instance in which the tube diameter of the above description is measured using ultrasonic arrays set to a frequency for obtaining the frequency shift amount, there are instances in which the detection accuracy decreases. In contrast, determining the tube diameter using diameter-measuring ultrasonic arrays that can output ultrasonic waves having a high frequency, at which the efficiency of reflection at the tube wall is high, makes it possible to perform measurement of the tube diameter to an even higher degree of accuracy. The fluid pressure of the measured fluid can thereby be determined in a more accurate manner.

A biological testing device according to another aspect of the present invention includes the measuring device as described above, and an acoustic matching part covering a surface of the ultrasonic arrays in the ultrasonic sensor, the acoustic matching part having an acoustic impedance that is equivalent to an acoustic impedance of a living body.

The acoustic matching part is one in which ultrasonic waves transmitted from the ultrasonic sensor passes through the acoustic matching part and reaches the interior of the body, and ultrasonic waves reflected in the body passes through the acoustic matching part and reaches the ultrasonic sensor. The acoustic impedance of the acoustic matching part and the acoustic impedance of the body are not required to completely coincide. In other words, “the acoustic impedance of the acoustic matching part being equivalent to the acoustic impedance of the body” refers to the acoustic impedances being in a range within which transmission/reception of ultrasonic waves is possible between the interior of the body and the ultrasonic sensor.

According to the aspect described above, the biological testing device includes an acoustic matching part arranged on each of the ultrasonic arrays of the measuring device. Therefore, placing the acoustic matching part in close contact with the skin of the body and transmitting ultrasonic waves from the ultrasonic sensor makes it possible to send out ultrasonic waves into the body, and receiving ultrasonic waves that has been reflected at, e.g., a blood vessel or another organ in the body makes it possible to perform an examination of the organ. For example, in an instance in which the organ on which measurement is performed is a blood vessel, measuring the frequency shift amount makes it possible to measure the flow velocity of the blood flowing through the blood vessel, or a similar variable.

As described above, the measuring device is able to readily measure an appropriate frequency shift amount using a simple configuration; therefore, a biological testing device comprising a measuring device of such description can be simplified in terms of configuration, and used to readily perform biological examination.

A flow velocity measuring method according to another aspect of the present invention is a method for measuring a flow velocity of a measured fluid using an ultrasonic sensor having a plurality of ultrasonic arrays arranged on a substrate, each of the ultrasonic arrays having a linear array structure in which a plurality of ultrasonic elements are arranged along a linear scanning direction to transmit/receive ultrasonic waves so that linear scanning directions are different from each other for the ultrasonic arrays. The flow velocity measuring method includes: controlling a transmission angle at which ultrasonic waves are transmitted from each of the ultrasonic arrays and performing transmission of ultrasonic waves from the ultrasonic arrays and reception of reflected ultrasonic waves; calculating, for each of the ultrasonic arrays, a frequency shift amount based on a reception signal outputted from each of the ultrasonic arrays, the frequency shift amount being a difference between a frequency of transmitted ultrasonic waves and a frequency of received ultrasonic waves; obtaining a maximum frequency shift amount, which is the largest of the frequency shift amounts calculated for each of the ultrasonic arrays in the calculating of the frequency shift amount; and calculating the flow velocity of the measured fluid based on the frequency of transmitted ultrasonic waves, the maximum frequency shift amount, and a direction of movement of the measured fluid.

According to the aspect described above, the frequency-shift-amount-calculating step calculates frequency shift amounts based on the reception signal from each of the ultrasonic arrays, and the maximum-shift-amount-obtaining step obtains the maximum frequency shift amount, which is the largest in value of these frequency shift amounts. Then, the flow-velocity-measuring step calculates the flow velocity, based on the direction of movement of the measured fluid measured by the movement-direction-detecting step, the maximum frequency shift amount, and the frequency of ultrasonic waves transmitted from the ultrasonic array.

As with the aspects described in the foregoing, a flow velocity measuring method of such description makes it possible to readily obtain the maximum frequency shift amount corresponding to an optimum ultrasound incidence angle without the user adjusting the position of the ultrasonic sensor, and to readily determine the flow velocity computationally using the maximum frequency shift amount.

A pressure measuring method according to another aspect of the present invention includes: measuring the flow velocity of the measured fluid by the flow velocity measuring method as described above; obtaining a flow path diameter of the measured fluid; and calculating the pressure of the measured fluid based on the flow path diameter and the flow velocity of the measured fluid.

According to the aspect described above, as with the aspects described in the foregoing, it is possible to readily obtain the maximum frequency shift amount corresponding to an optimum ultrasound incidence angle, to readily determine the flow velocity computationally using the maximum frequency shift amount; therefore, it is also possible to readily determine the fluid pressure of the measured fluid computationally.

### BRIEF DESCRIPTION OF THE DRAWINGS

Referring now to the attached drawings which form a part of this original disclosure:

FIGS. 1A and 1B are perspective views showing an overview of a biological testing device according to a first embodiment of the present invention, where FIGS. 1A and 1B are views showing a front surface side and a back surface side of the biological testing device, respectively;

FIG. 2 is a block diagram showing an overview of a configuration of the biological testing device according to the first embodiment;

FIG. 3 is a top view showing an overview of a configuration of an ultrasonic sensor according to the first embodiment;

FIG. 9A is a top view showing an enlargement of an ultrasonic array according to the first embodiment, and FIG. 4B is a cross-section view of the same;

FIG. 5 is a diagram showing the transmission angle of ultrasonic waves when a drive signal inputted into each of the ultrasonic elements (1) through (4) is inputted sequentially with a delay of Δt;

FIG. 6 is a diagram showing a scan area for a single ultrasonic array according to the first embodiment;

FIG. 7 is a diagram showing the beam shape of ultrasonic waves transmitted from an ultrasonic transducer according to the first embodiment;

FIG. 8 is a diagram showing a scan area of each of the ultrasonic arrays in the ultrasonic sensor according to the first embodiment;

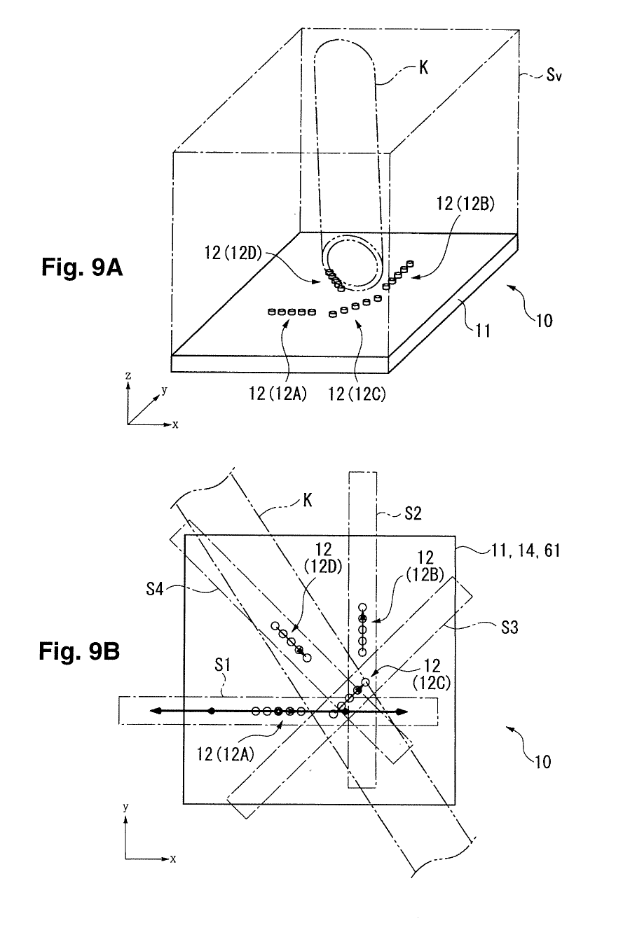

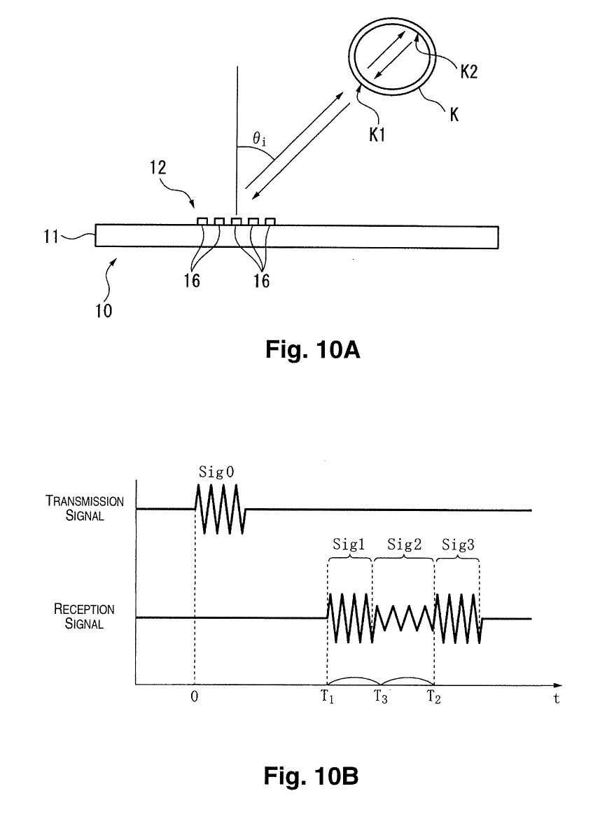

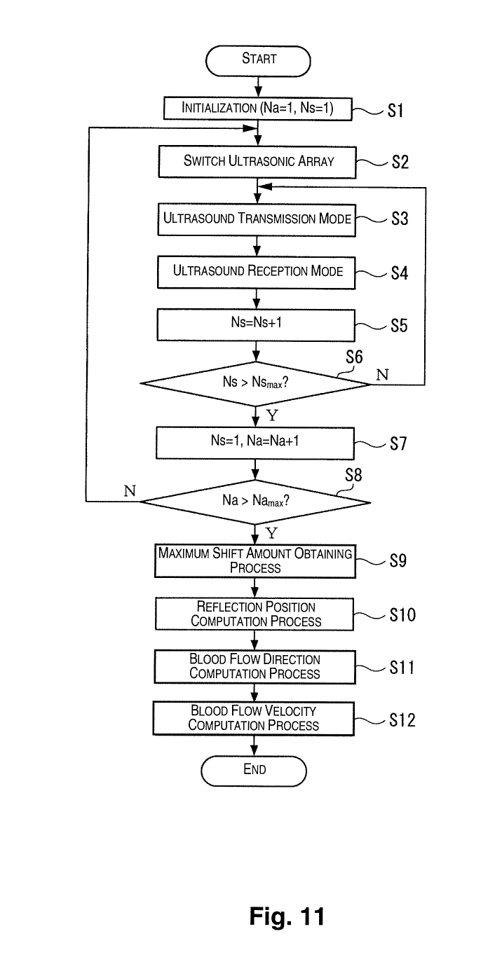

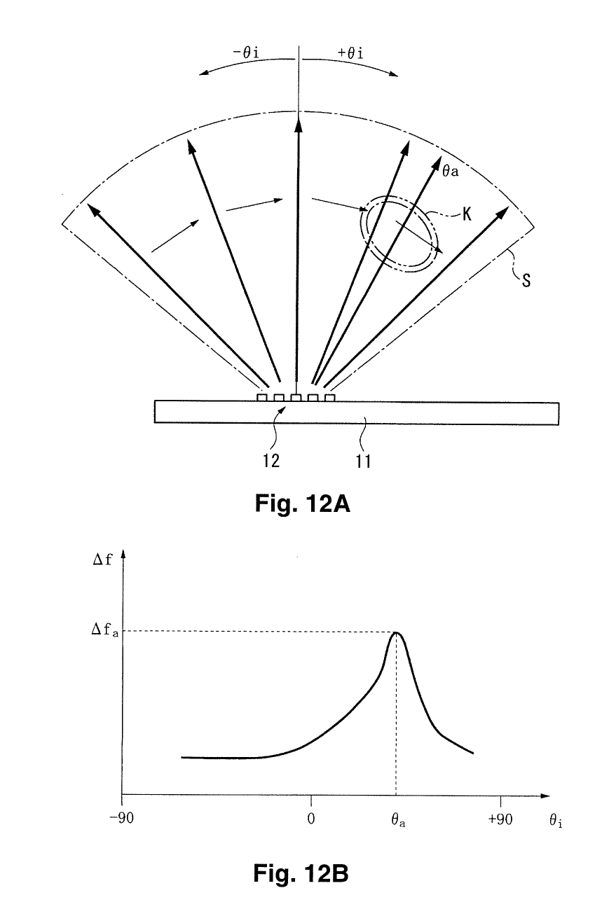

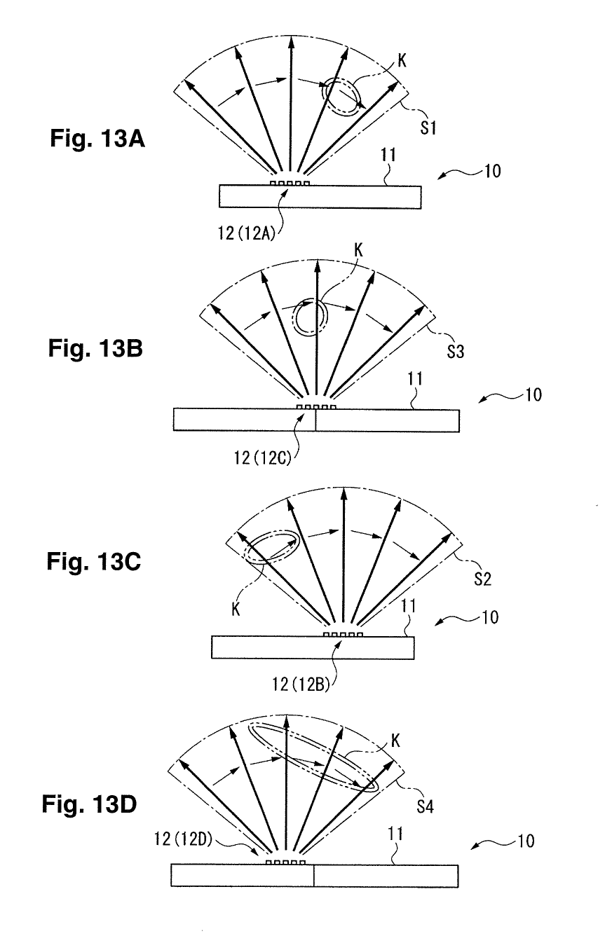

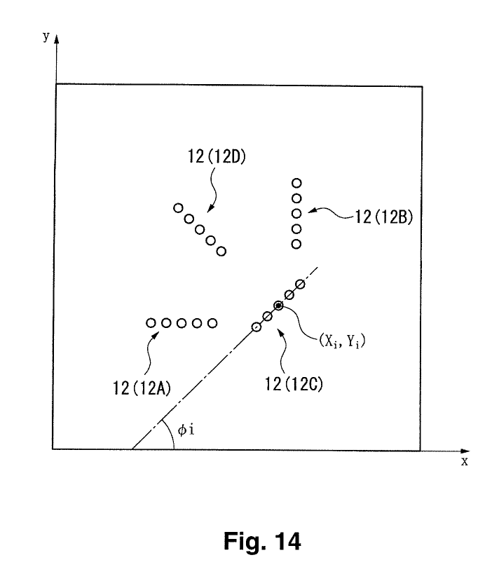

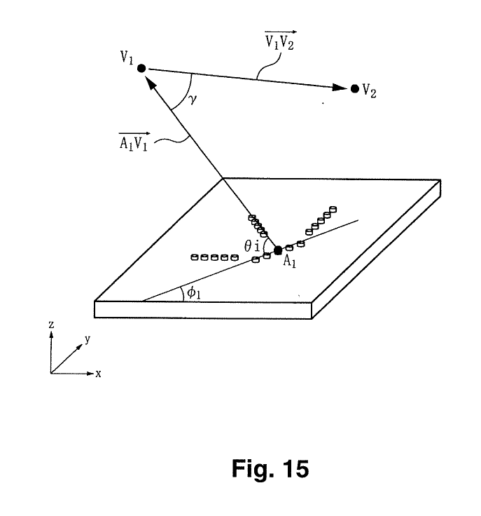

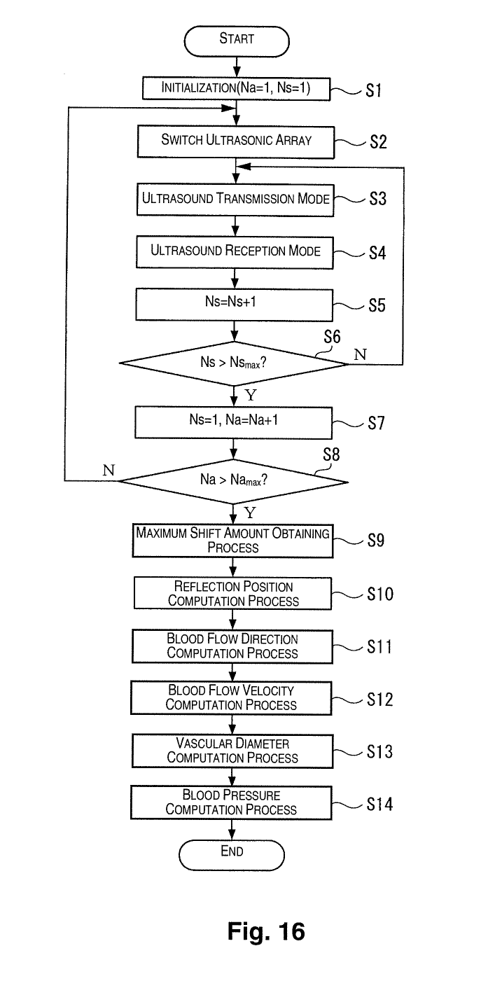

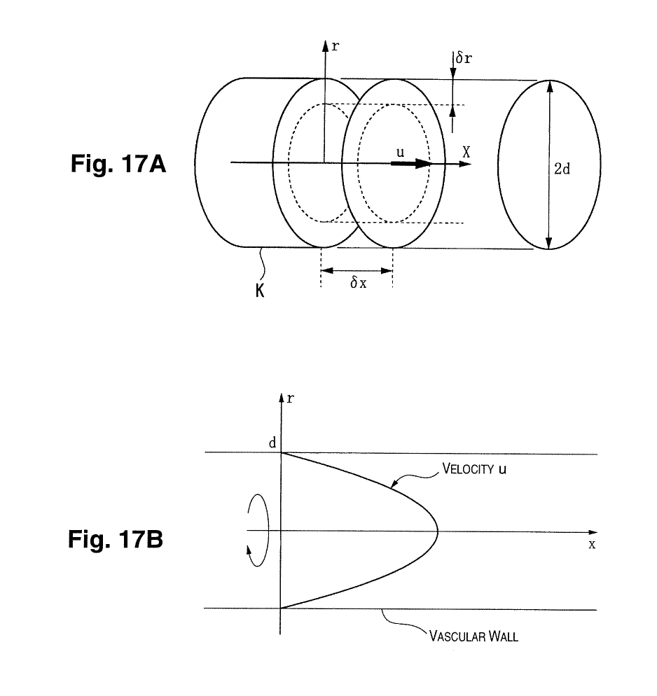

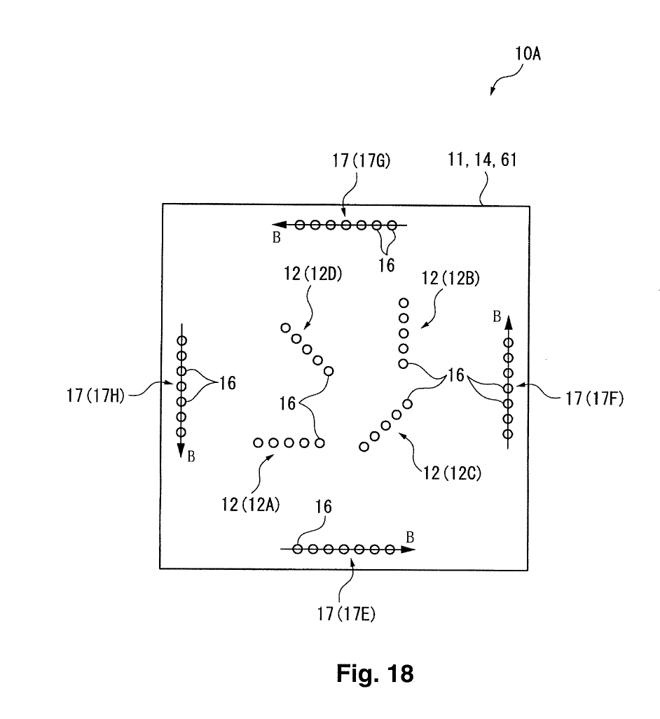

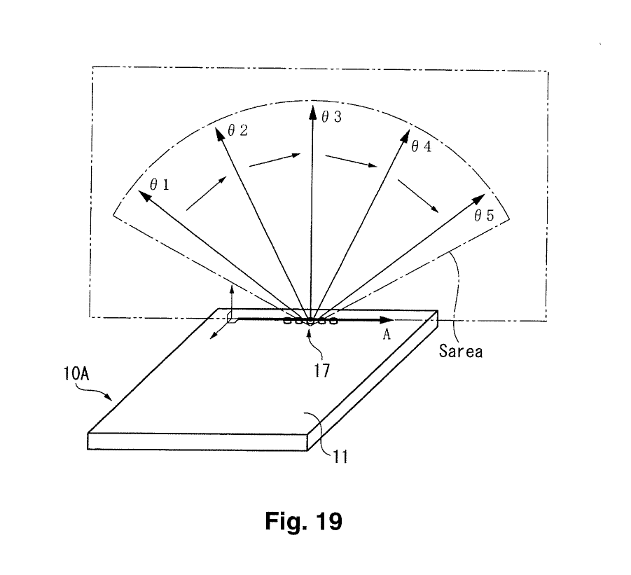

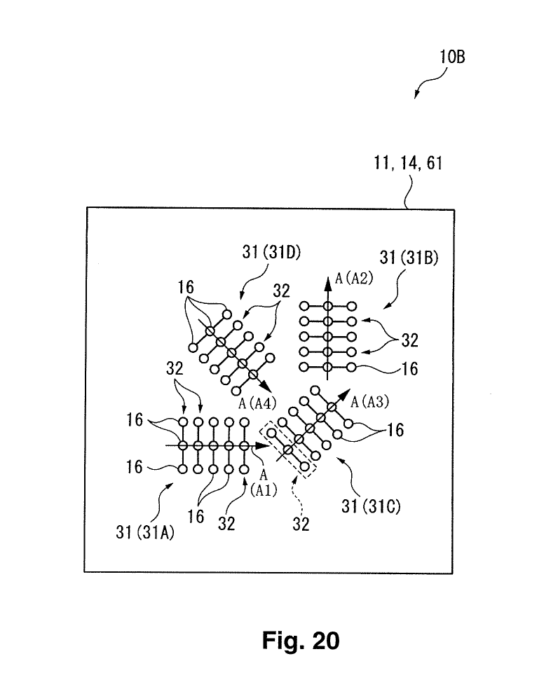

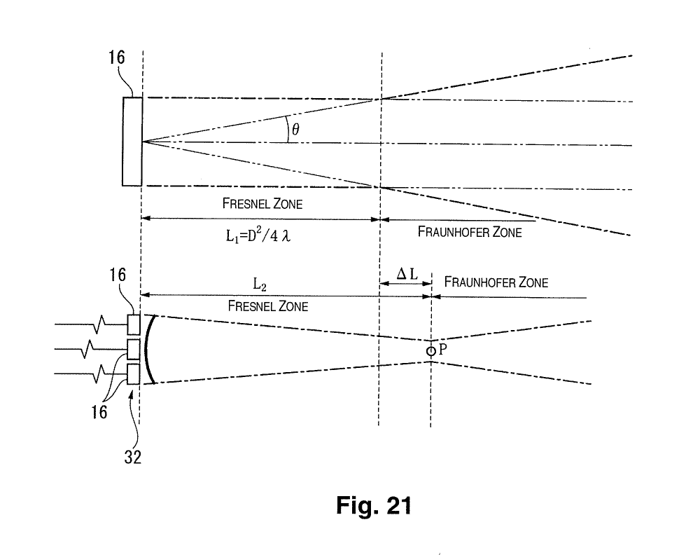

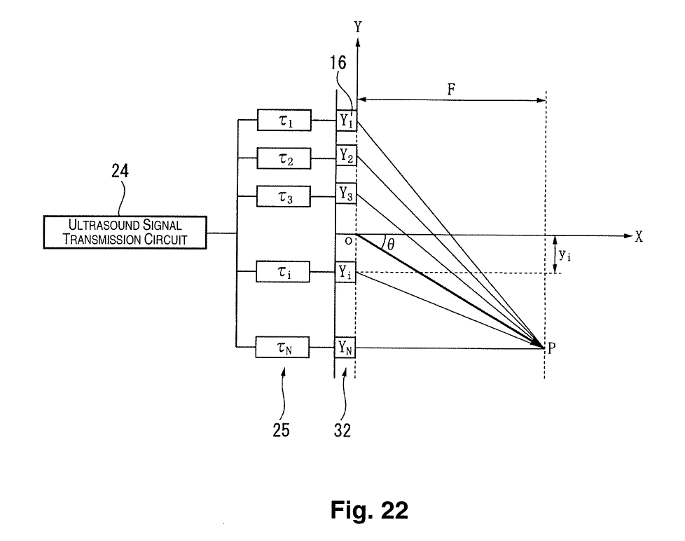

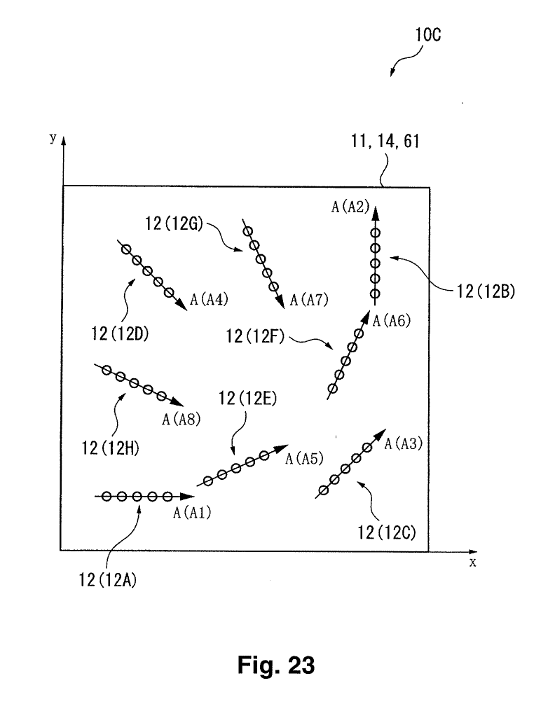

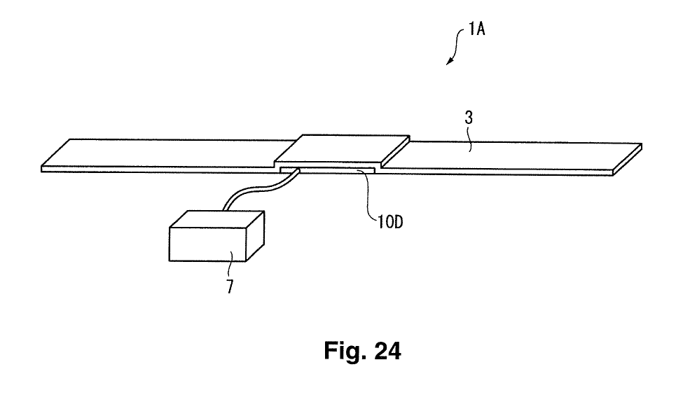

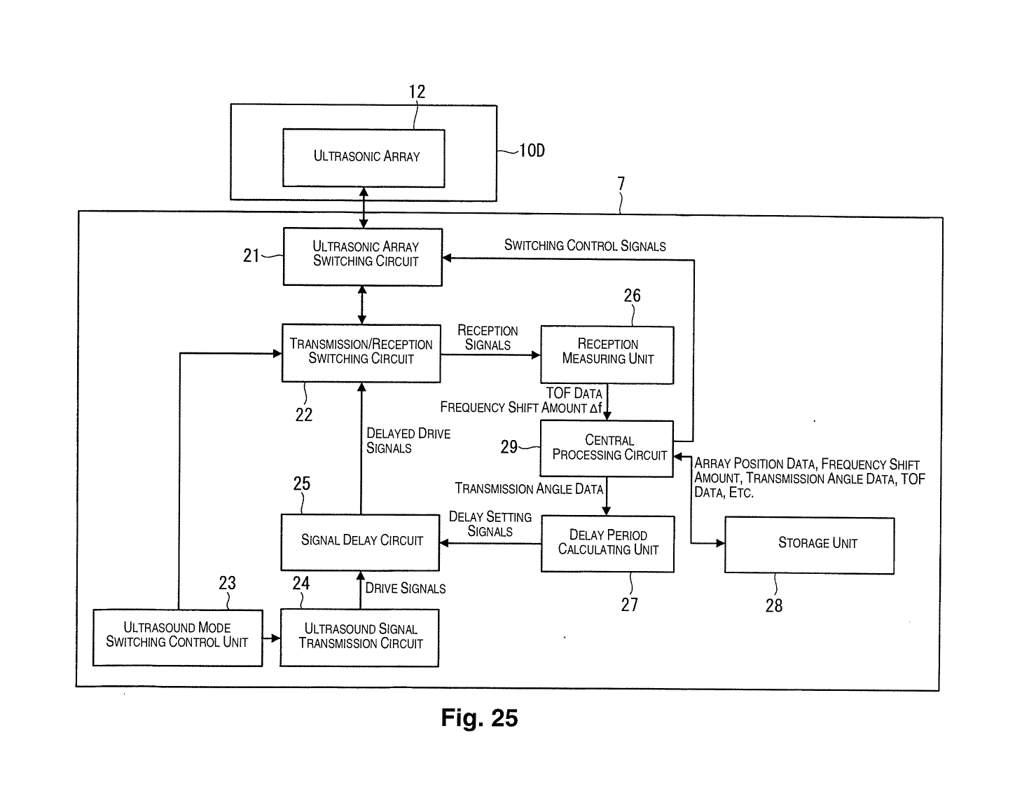

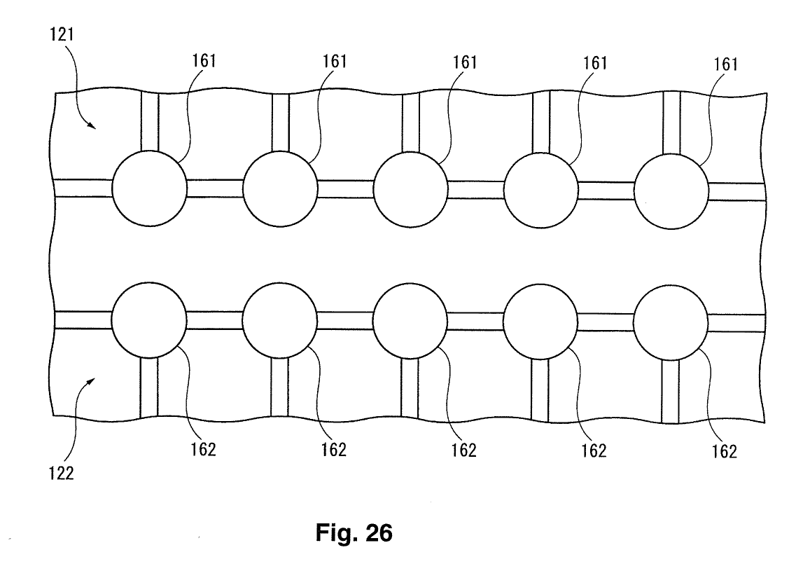

FIGS. 9A and 9B are diagrams showing an example of an instance in which a blood vessel is present in the scan area of the ultrasonic sensor, where FIG. 9A is a perspective view and FIG. 9B is a plan view;

FIGS. 10A and 10B are diagrams used to describe a method of measuring the reflection position;

FIG. 11 is a flow chart showing a blood flow measuring process for the biological testing device according to the present invention;

FIG. 12A is a diagram showing a scan area in an instance in which the transmission angle of ultrasonic waves is varied with regards to a single ultrasonic array, and FIG. 12B is a diagram showing a variation in the frequency shift amount obtained from the reception signal;

FIGS. 13A to 13D are diagrams showing an example of intersecting positions of a blood vessel that intersects each of the scan areas;

FIG. 14 is a diagram showing position data with regards to the ultrasonic array;

FIG. 15 is a diagram showing a model for computation of the direction of blood flow;

FIG. 16 is a flow chart showing a blood pressure measuring process for a biological testing device according to a second embodiment of the present invention;

FIG. 17A is a schematic diagram showing an enlargement of a part of a blood vessel, and FIG. 17B is a diagram showing a distribution of velocity of blood in a blood vessel;

FIG. 18 is a top view showing the plane of the substrate of an ultrasonic sensor of a biological testing device according to the third embodiment;

FIG. 19 is a diagram showing a scan area of a single position-measuring ultrasonic array;

FIG. 20 is a top view of a substrate of an ultrasonic sensor of the biological testing device according to the fourth embodiment;

FIG. 21 is a diagram showing the difference between the Fresnel zone of ultrasonic waves outputted by a single ultrasonic transducer (top) and the Fresnel zone of an ultrasonic array according to the second embodiment;

FIG. 22 is a diagram showing a state in which ultrasonic waves transmitted from a plurality of ultrasonic transducers is focused at a predetermined single point;

FIG. 23 is a top view showing an overview of a configuration of an ultrasonic sensor according to the fifth embodiment of the present invention;

FIG. 24 is a perspective view showing an overview of a biological examination system according to the sixth embodiment of the present invention;

FIG. 25 is a block diagram showing an overview of a configuration of the biological examination system according to the sixth embodiment; and

FIG. 26 is a top view showing an enlargement of an ultrasonic array according to another embodiment.

### DETAILED DESCRIPTION OF EXEMPLARY EMBODIMENTS

### First Embodiment

A biological testing device comprising a measuring device having an ultrasonic sensor according to the first embodiment of the present invention will now be described with reference to the accompanying drawings.

### 1. Overall Configuration of Biological Testing Device

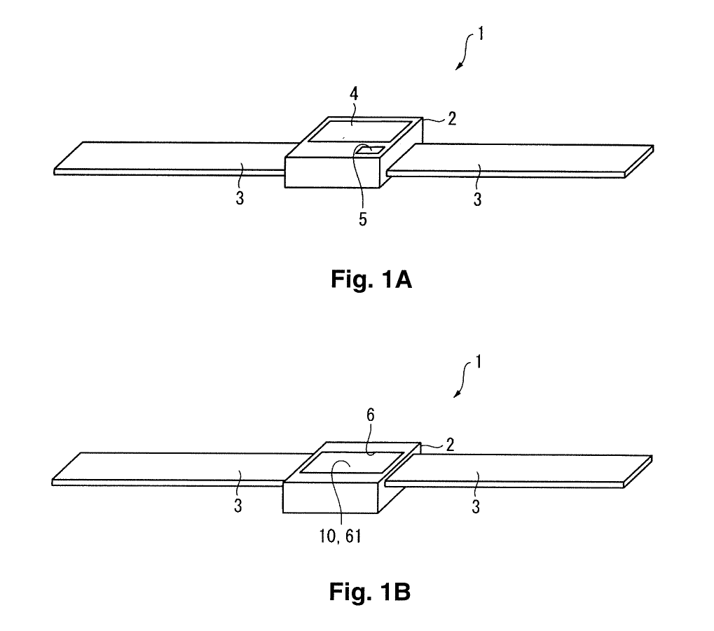

FIGS. 1A and 1B are perspective views showing an overview of a biological testing device according to a first embodiment, where FIGS. 1A and 1B are views showing a front surface side and a back surface side of the biological testing device, respectively.

In FIGS. 1A and 1B, a biological testing device 1 is a device for using ultrasound to measure the state of a blood vessel, and specifically, a device for measuring the flow velocity of blood flowing in the blood vessel (i.e., blood flow velocity), the blood being a fluid to be measured. As shown in FIGS. 1A and 1B, the biological testing device 1 comprises a main device body 2, and a band 3 connected to the main device body 2. The biological testing device 1 is worn on the body by tightening the band 3 in a state in which the reverse surface is in close contact with the body, and it becomes possible to monitor and measure the state of the blood vessel over, e.g., 24 hours.

### 2. Configuration of Main Device Body

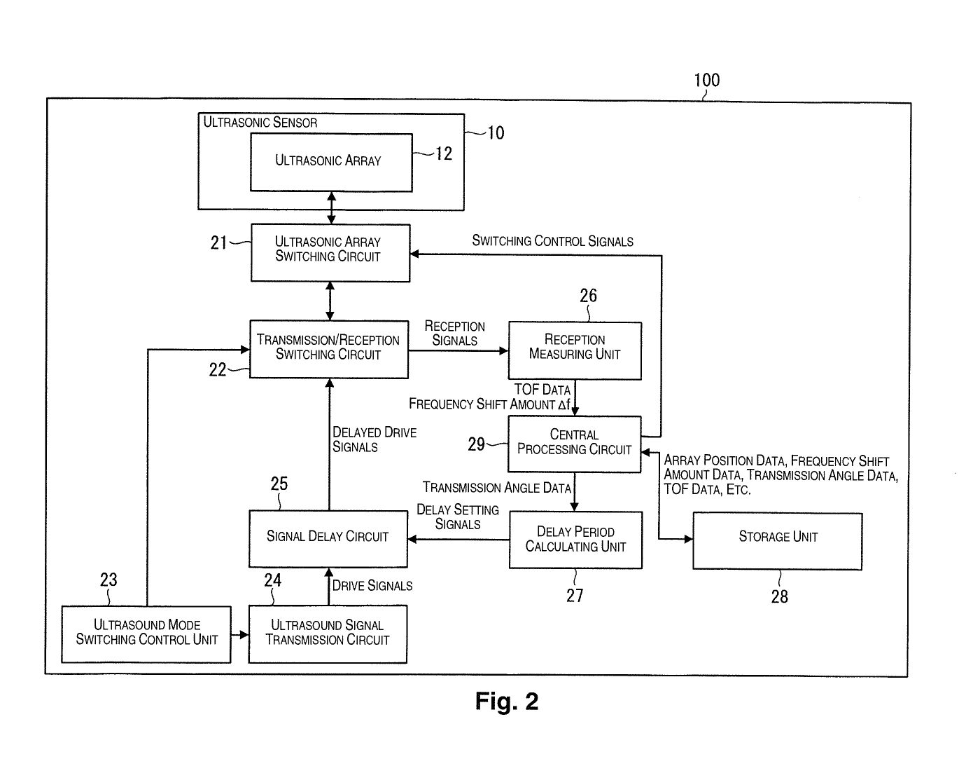

A display part 4 for showing measurement results, an operating part 5 for operating the biological testing device 1, and other components are provided on a front surface side of the main device body 2 of the biological testing device 1, as shown in FIG. 1A. A sensor window 6 is formed on a reverse surface side of the main device body 2, and an acoustic matching part 61 is arranged from the sensor window 6. A measuring device 100 (see FIG. 2) comprising an ultrasonic sensor 10 (see FIG. 2) is provided in the interior of the main device body 2. The ultrasonic sensor 10 is provided integrally with the acoustic matching part 61.

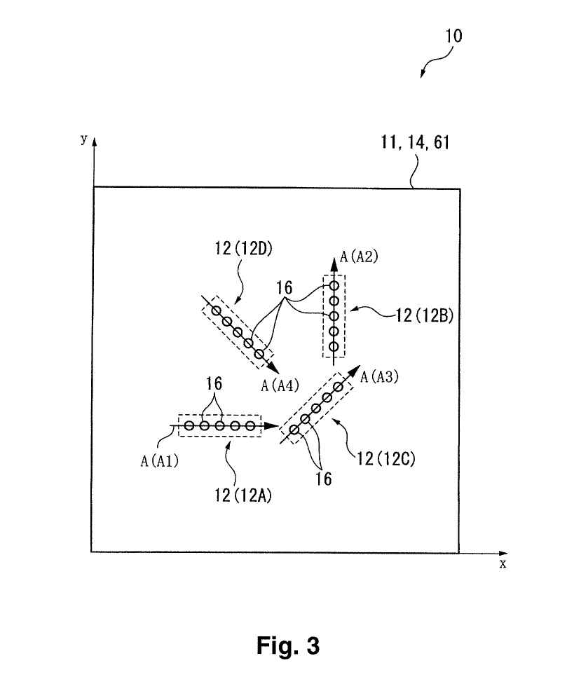

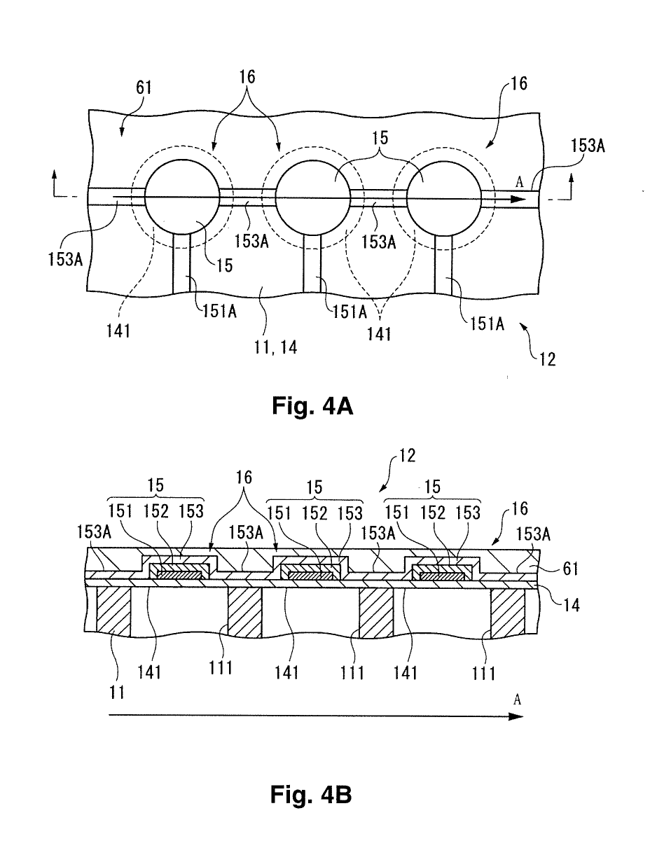

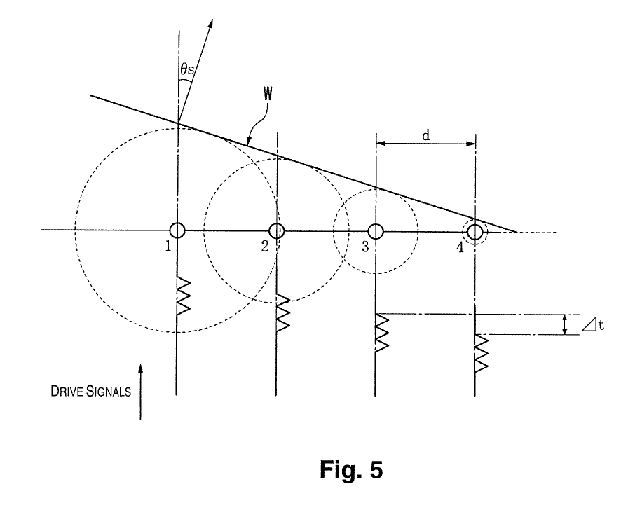

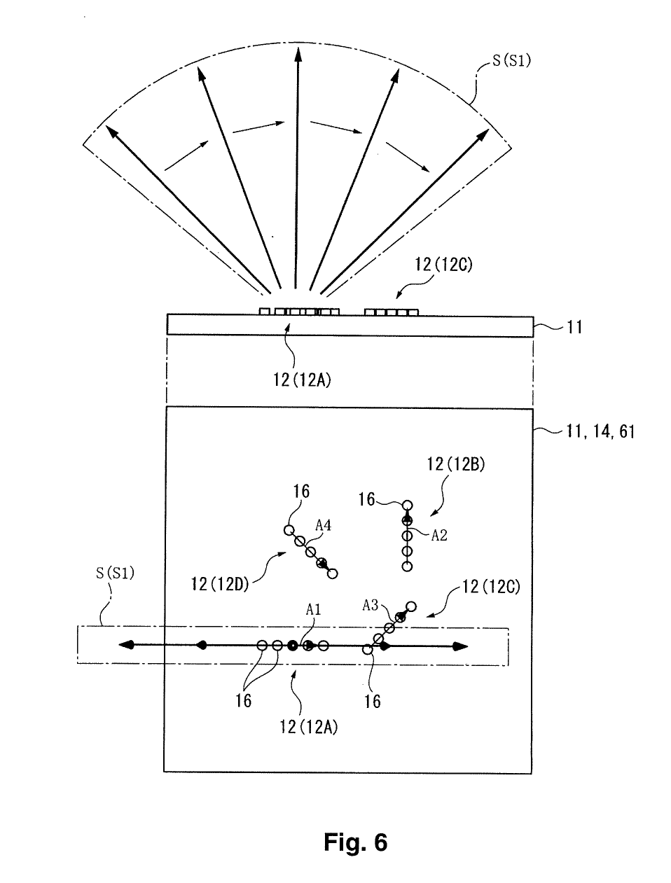

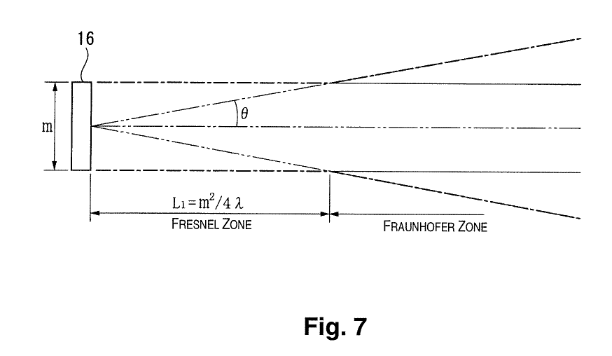

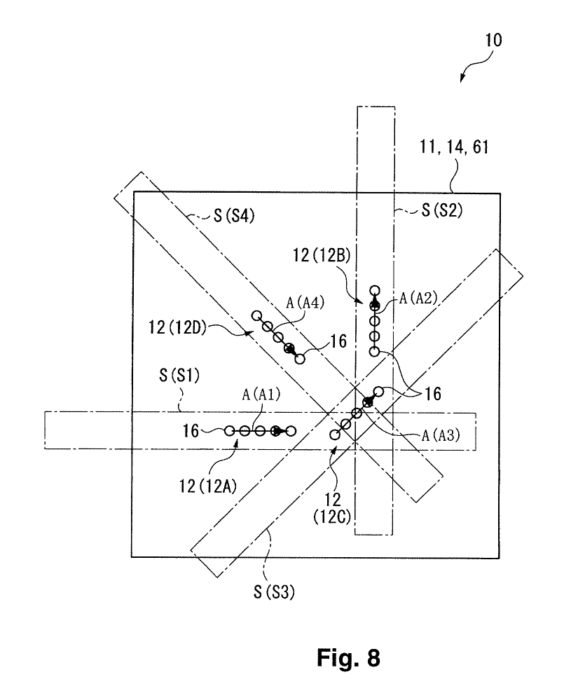

The acoustic matching part 61 is formed from, e.g., silicone rubber or another material whose acoustic impedance is substantially equivalent to that of the body. The acoustic matching part 61 is a layer for protecting each ultrasonic array 12 (described further below; see FIG. 3), wiring patterns formed on a support film 14 (see FIG. 3), and other structures from external pressure, and is formed from e.g., silicone rubber.

In a biological testing device 1 of such description, the acoustic matching part 61 is placed in close contact with the body when the state of the blood vessel in the body is measured. When ultrasound is sent out from the ultrasonic sensor 10 towards the acoustic matching part 61 in this state, the ultrasonic waves are propagated from the acoustic matching part 61 to the interior of the body. Ultrasonic waves that have been reflected by a blood vessel or another tissue in the body passes through the acoustic matching part 61 and enters into the ultrasonic sensor 10.

FIG. 2 is a block diagram showing an overview of a configuration of the measuring device 100 of the biological testing device 1 according to the present embodiment.

As shown in FIG. 2, the measuring device 100 is configured so as to include the ultrasonic sensor 10, an ultrasonic array switching circuit 21, a transmission/reception switching circuit 22, an ultrasound mode switching control unit 23, an ultrasound signal transmission circuit 24, a signal delay circuit 25, a reception measuring unit 26, a delay period calculating unit 27, a storage unit 28, and a central processing circuit 29.

### 2-1. Configuration of Ultrasonic Sensor

FIG. 3 is a top view showing an overview of a configuration of an ultrasonic sensor according to the first embodiment.

As shown in FIG. 3, the ultrasonic sensor 10 comprises a rectangular substrate 11. Also, ultrasonic arrays 12 (12A, 12B, 12C, 12D) are provided at a substantially center part of the substrate 11 with respect to a plan view when the substrate 11 is viewed from the thickness direction of the substrate 11. More specifically, the ultrasonic sensor 10 comprises the substrate 11 and the support film 14 (see FIGS. 4A and 4B) that is formed in layered fashion on the substrate 11. The acoustic matching part 61 described above is formed so as to cover the ultrasonic arrays 12 from above.

FIG. 4A is a top view showing an enlargement of an ultrasonic array according to the first embodiment, and FIG. 4B is a cross-section view of the same.

The ultrasonic arrays 12 are arranged at a central part of each of the sides of the substrate 11 as described above. Each of the ultrasonic arrays 12 comprises ultrasonic transducers 16, each of which transducers comprising a diaphragm 141 and a piezoelectric body 15.

Specifically, as shown in FIGS. 4A and 4B, the substrate 11 is formed as, e.g., a rectangle. An opening part 111 for forming the diaphragm 141 of each of the ultrasonic transducers 16 of the ultrasonic arrays 12 is formed in a plurality at the center part of the substrate 11.

When x-y axes are configured on the substrate 11 with one of the vertices of the rectangle being the origin as shown in FIG. 3, the ultrasonic array 12A has a linear scanning direction A1 that is parallel to the x-axis, and comprises a plurality of ultrasonic transducers 16 arranged along the linear scanning direction A1. The ultrasonic array 12B has a linear scanning direction A2 that is parallel to the y-axis, and comprises a plurality of ultrasonic transducers 16 arranged along the linear scanning direction A2. The ultrasonic array 12C has a linear scanning direction A3 that is inclined at an angle of 45° with respect to the x-axis, and comprises a plurality of ultrasonic transducers 16 arranged along the linear scanning direction A3. The ultrasonic array 12D has a linear scanning direction A4 that is inclined at an angle of 135° with respect to the x-axis, and comprises a plurality of ultrasonic transducers 16 arranged along the linear scanning direction A4. Each of the opening parts 111 is formed with respect to the position at which each of the ultrasonic transducers 16 is arranged on the substrate 11.

Also, as described above, the support film 14 is laminated on the substrate 11. Each of the opening parts 111 described above is blocked by the support film 14. A region of the support film 14 that blocks the opening part 111 forms the diaphragm 141. The piezoelectric body 15 is provided on the diaphragm 141.

The present embodiment shows an example in which a common substrate 11 is used for the ultrasonic sensor 10 and the ultrasonic arrays 12. However, other configurations are also possible; e.g., one in which an array substrate forming the ultrasonic arrays 12 is separately arranged on a substrate 11 forming the ultrasonic sensor 10.

The ultrasonic sensor 10 and the ultrasonic transducers 16 according to the present embodiment will now be described more specifically. The substrate 11 is formed from silicon (Si) or another semiconductor-forming material that can be readily machined by etching or a similar procedure. Also, the opening part 111 formed on the substrate 11 is formed as, e.g., a circle with respect to the plan view. Although the shape of the opening part 111 with respect to the plan view is a circle in the present example, a circle is not provided by way of limitation. The opening part 111 may be formed so as to be, e.g., a rectangle or another shape with respect to the plan view, according to the deflection balance of the diaphragm 141, or the vibration stability of the diaphragm 141 when caused by the piezoelectric body 15 to vibrate.

The support film 14 is formed on the substrate 11 in a state of blocking the opening part 111. The support film 14 is configured from, e.g., a two-layer structure of a SiO₂ film and a ZrO₂ layer.

In an instance in which the substrate 11 is an Si substrate, the SiO₂ layer can be formed by performing thermal oxidation treatment on the surface of the substrate. The ZrO₂ layer is formed by, e.g., sputtering or performing another method on the SiO₂ surface. In an instance in which, e.g., PZT is used as a piezoelectric film 152 described further below, the ZrO₂ layer is a layer for preventing Pb, which forms PZT, from diffusing into the SiO₂ layer. The ZrO₂ layer also has an effect of improving the deflection efficiency of the piezoelectric film 152 in response to strain.

The piezoelectric body 15 comprises a lower electrode 151 laminated on a layer above the support film 14, the piezoelectric film 152 formed on the lower electrode 151, and an upper electrode 153 formed on the piezoelectric film 152.

A lower electrode wire 151A extending on the support film 14 along an orthogonal-to-scan direction, which is orthogonal to the linear scanning direction A, is connected to the lower electrode 151 as shown, e.g., in FIG. 4A. The lower electrode wire 151A is provided independently of each other in relation to each of the ultrasonic transducers 16.

An upper electrode wire 153A extending along the linear scanning direction A on the support film 14 is connected to the upper electrode 153. The upper electrode wire 153A is a common electrode wire for a single ultrasonic array 12. Specifically, the upper electrode wire 153A is connected to the upper electrode 153 of adjacent ultrasonic transducers 16 as shown in FIGS. 4A and 4B, and, at an end part, is connected to, e.g., a ground. The upper electrode 153 of each of the ultrasonic transducers 16 is thereby earthed.

Although the present example shows a configuration in which the upper electrode wire 153A is connected to ground as a common electrode wire for the ultrasonic arrays 12, and the lower electrode wires 151A are formed independently of each other, whereby each of the ultrasonic transducers 16 can be individually driven, a configuration is also possible in which, e.g., the lower electrode wire 151A is connected to ground as a common electrode wire and the upper electrode wires 153A are formed independently of each other.

The material forming the lower electrode 151, the upper electrode 153, the lower electrode wire 151A, and the upper electrode wire 153A is an electrically conductive metal film; a laminated film comprising a plurality of layers of metal film may also be used. In the present embodiment, a Ti/Ir/Pt/Ti laminated film is used as the lower electrode 151 and the lower electrode wire 151A, and an Ir film is used as the upper electrode 153 and the upper electrode wire 153A.

The piezoelectric film 152 is formed by, e.g., forming PZT (i.e., lead zirconate titanate) into the shape of a film. Although PZT is used for the piezoelectric film 152 in the present embodiment, any material may be used as long as the material is capable of contracting in the in-plane direction upon being subjected to a voltage. For example, lead titanate (PbTiO₃), lead zirconate (PbZrO₃), lead lanthanum titanate ((Pb, La) TiO₃), or a similar material may also be used.

In an ultrasonic transducer 16 of such description, a voltage is applied to the lower electrode 151 and the upper electrode 153, whereby the piezoelectric film 152 expands/contracts in the in-plane direction. In this instance, a surface on one side of the piezoelectric film 152 is joined to the support film 14 with the lower electrode 151 interposed therebetween, and the upper electrode 153 is formed on a surface on the other side. Since no other layer is formed in layered fashion on the upper electrode wire 153A, the side of the piezoelectric film 152 towards the support film 14 expands/contracts less readily, and the side towards the upper electrode 153 expands/contracts more readily. Therefore, when a voltage is applied to the piezoelectric film 152, a deflection is generated so as to be convex towards the opening part 111, and the diaphragm 141 is caused to deflect. Therefore, applying an AC voltage to the piezoelectric film 152 causes the diaphragm 141 to vibrate in the direction of film thickness, and this vibration of the diaphragm 141 causes ultrasonic waves to be transmitted.

In an instance in which ultrasonic waves are received by the ultrasonic transducer 16, when ultrasound is inputted into the support film 14, the support film 14 vibrates in the direction of film thickness. In the ultrasonic transducer 16, this vibration of the diaphragm 141 generates a potential difference between the surface of the piezoelectric film 152 towards the lower electrode 151 and the surface towards the upper electrode 153. A reception signal (i.e., an electrical current) that corresponds with the amount of displacement of the piezoelectric film 152 is outputted from the upper electrode 153 and the lower electrode 151.

In each of the ultrasonic arrays 12 in which ultrasonic transducers 16 of such description are arranged in a plurality along the linear scanning direction A, delaying and staggering the timing at which ultrasonic waves are transmitted from each of the ultrasonic transducers 16 makes it possible to transmit ultrasound planar waves in the desired direction.

FIG. 5 is a diagram showing the direction of transmission (i.e., transmission angle) of ultrasound when a drive signal inputted into each of the ultrasonic elements (1) through (4) is inputted sequentially with a delay of Δt.

When ultrasonic waves are transmitted from each of the ultrasonic transducers 16, a compounded wave front W in which the individual ultrasonic beams strengthen each other is formed, and the compounded wave front W propagates. If, as shown in FIG. 5, the drive signal inputted into each of the ultrasonic elements (1) through (4), which have been arranged at intervals equal to d, is delayed by Δt, the wave front of ultrasonic waves transmitted from the ultrasonic transducer 16 into which a drive signal has been inputted earlier has a different phase to the wave front of ultrasonic waves transmitted from the ultrasonic transducer 16 into which a drive signal has been inputted later. Therefore, the compounded wave front W propagates at an incline with respect to the linear scanning direction A.

In this instance, if θs represents the transmission angle between the direction of propagation of the compounded wave front W and the orthogonal-to-scan direction, which is orthogonal to the linear scanning direction A; and c represents the speed of sound, the relationship shown in the following Equation (2).

$$\begin{matrix}Equation ( 2 ) & \\ Δ t = \frac{d {θ}_{s}}{c} & ( 2 )\end{matrix}$$

FIG. 6 is a diagram showing a scan area for a single ultrasonic array 12. FIG. 7 is a diagram showing the beam shape of ultrasonic waves transmitted from an ultrasonic transducer 16. FIG. 8 is a diagram showing a scan area of each of the ultrasonic arrays 12 in the ultrasonic sensor 10. In the following description, the acoustic matching part 61 is not shown in schematic diagrams of the sensor cross section such as the top diagram in FIG. 6 in order to ease understanding of the description.

In each of the ultrasonic arrays 12, as described above, the timing of the drive signal inputted into each of the ultrasonic transducers 16 is delayed, thereby making it possible to vary the ultrasound transmission angle. Since each of the ultrasonic arrays 12 has a linear array structure (i.e., a one-dimensional array structure), the ultrasound transmission angle is restricted to a scan plane that is disposed along the linear scanning direction A and orthogonal to the substrate 11 as shown in FIG. 6, and the transmission angle cannot be varied in a direction that intersects the scan plane.

Ultrasonic waves transmitted from the diaphragm 141 of each of the ultrasonic transducers 16, which have a finite area, has a beam shape as shown in FIG. 7 (shown by dashed-dotted lines in FIG. 7). The range extending from the diaphragm 141 to a distance of m2/4λ represents the Fresnel zone, where m represents the diameter of the diaphragm 141 of the ultrasonic transducer 16, and ultrasonic waves having a wavelength of λ is transmitted. In the Fresnel zone, it is possible to cause the ultrasonic waves to propagate as substantially planar waves. In the Fraunhofer zone, which represents the range in excess of the Fresnel zone, the wave front of the ultrasonic waves forms a spherical shape, and propagates in dispersed manner. In an instance in which ultrasound is used to measure the position of the blood vessel, in the Fraunhofer zone, the ultrasonic waves disperse, and it is therefore not possible to obtain an accurate position information. Therefore, blood vessel within the Fresnel zone is detected by ultrasound.

Thus, a scan area S, in which the position of the blood vessel can be measured using a single ultrasonic array 12, describes a fan-shaped range that is coplanar with respect to a scan plane that is disposed along the linear scanning direction A and orthogonal to the substrate 11, and in which the distance from the ultrasonic array 12 is within the range of the Fresnel zone (i.e., a range extending to a distance of m2/4λ from the ultrasonic array 12), as shown in FIG. 6.

In the present example, the thickness dimension of the ultrasonic transducers 16 in the orthogonal-to-scan direction is sufficiently small, and the scan area S with respect to the ultrasonic array 12 is therefore considered to be coplanar with respect to the scan plane. However, in an instance in which, e.g., the ultrasonic transducers 16 are formed so as to have an elongated shape in the orthogonal-to-scan direction, the scan area S is a volume region having a width dimension equal to the dimension of the ultrasonic transducers 16 in the elongated direction.

In the ultrasonic sensor 10 according to the present embodiment, ultrasonic arrays 12 of the above description are arranged on the central part of the substrate 11 so that each of the linear scanning directions Al through A4 are disposed so as to differ from one another. Therefore, the four ultrasonic arrays 12 result in a distribution of scan areas S (S1 through S4), each having a respectively different plane direction.

FIGS. 9A and 9B are diagrams showing an example of an instance in which a blood vessel is present in the scan area of the ultrasonic sensor, where FIG. 9A is a perspective view and FIG. FIG. 9B is a plan view. In the following description, the acoustic matching part 61 is not shown in schematic perspective views of the sensor such as FIG. 9A in order to ease understanding of the description.

In an ultrasonic sensor 10 of such description, in an instance in which a blood vessel K passes through a scannable region Sv directly below the ultrasonic sensor 10, the blood vessel K passes through at least two scan areas S out of each of the scan areas S of the four ultrasonic arrays 12. Therefore, transmitting ultrasonic waves from these ultrasonic arrays 12 and receiving ultrasonic waves reflected by the blood vessel K makes it possible to obtain the position of the blood vessel K and the frequency shift amount.

### 2-2. Configuration of Ultrasonic Array Switching Circuit

Next, a description will be given for other structures in the main device body 2 with reference again to FIG. 2.

The ultrasonic array switching circuit 21 is a circuit for switching the ultrasonic array 12 that is to be driven from among the four ultrasonic arrays 12 provided to the ultrasonic sensor 10.

In the biological testing device 1 according to the present embodiment, while transmission/reception of ultrasonic waves is being performed from one ultrasonic array 12, outputting of drive signals to other ultrasonic arrays 12 and receiving of reception signals from other ultrasonic arrays 12 are not performed. It is thereby possible to avoid an adverse event where ultrasonic waves transmitted from another ultrasonic array 12 is received by the ultrasonic array 12 that is to be driven, and noise is detected; or where a reception signal is detected from an ultrasonic array 12 that is not to be driven.

The ultrasonic array switching circuit 21 comprises a terminal group that connects to, e.g., the lower electrode wire 151A and the upper electrode wire 153A of each of the ultrasonic arrays 12, and connects, based on a switching control signal for selecting an array inputted from the central processing circuit 29, the terminal group corresponding to each of the ultrasonic arrays 12 based on the switching control signal, to the transmission/reception switching circuit 22. Each of the terminal groups corresponding to the ultrasonic arrays 12 that are not to be driven may be configured so that, e.g., both of the lower electrode 151 and the upper electrode 153 are connected to ground so that the terminal groups are not driven.

### 2-3. Configuration of Transmission/Reception Switching Circuit

The transmission/reception switching circuit 22 is a switching circuit for switching the connection state, based on a mode switching signal inputted from the ultrasound mode switching control unit 23.

Specifically, in an instance in which a control signal for switching to the ultrasound transmission mode is inputted from the ultrasound mode switching control unit 23, the transmission/reception switching circuit 22 switches to a connection state in which a drive signal inputted from the signal delay circuit 25 can be outputted to the ultrasonic array switching circuit 21. On the other hand, in a state in which a control signal for switching to the ultrasound reception mode is inputted from the ultrasound mode switching control unit 23, the transmission/reception switching circuit 22 switches to a connection state in which the reception signal inputted from the ultrasonic array switching circuit 21 can be outputted to the reception measuring unit 26.

### 2-4. Configuration of Ultrasound Mode Switching Control Unit

The ultrasound mode switching control unit 23 switches between the ultrasound transmission mode, in which ultrasonic waves are transmitted from the ultrasonic arrays 12, and the ultrasound reception mode, in which ultrasonic waves are received by the ultrasonic arrays 12.

Specifically, when a control signal for commencing measurement of the state of the blood vessel is inputted from the central processing circuit, the ultrasound mode switching control unit 23 performs a process of switching to the ultrasound transmission mode. In this process, the ultrasound mode switching control unit 23 outputs, to the transmission/reception switching circuit 22, a control signal for switching to the transmission mode, and outputs a control signal for outputting a drive signal from the ultrasound signal transmission circuit 24. The ultrasound mode switching control unit 23 also identifies time measured by a time measuring part (not shown), and performs a process of switching to the ultrasound reception mode after a predetermined transmission time has elapsed in the ultrasound transmission mode. The transmission time is set to about a length in which, e.g., one or two cycles of burst waves are transmitted from the ultrasonic arrays 12. For the reception mode, the ultrasound mode switching control unit 23 outputs, to the transmission/reception switching circuit 22, a control signal for switching to the reception mode, and causes the transmission/reception switching circuit 22 to switch to a connection state in which the reception signal inputted from the ultrasonic arrays 12 can be inputted into the reception measuring unit 26.

The ultrasound mode switching control unit 23 performs the above process for a previously set number of repetitions. This number of repetitions is set as appropriate from the number of ultrasound transmission angles that have been set. For example, in an instance in which the transmission angle of ultrasonic waves is switched between five steps and the position of the blood vessel K is measured as shown in FIG. 6, the above process is repeated five times.

In an instance in which the position of the blood vessel could not be detected based on the reception signal, the above process may be repeated further.

### 2-5. Configuration of Ultrasound Signal Transmission Circuit

When a control signal for outputting the drive signal is inputted from the ultrasound mode switching control unit 23 while the transmission mode is enabled, the ultrasound signal transmission circuit 24 outputs, to the signal delay circuit 25, a drive signal (i.e., a driving voltage) for driving the ultrasonic transducers 16 of the ultrasonic arrays 12.

### 2-6. Configuration of Signal Delay Circuit

The signal delay circuit 25 forms a delay control unit of the present invention. When the drive signal in relation to each of the ultrasonic transducers 16 is inputted from the ultrasound signal transmission circuit 24, the signal delay circuit 25 applies a delay to the drive signal and outputs the drive signal to the transmission/reception switching circuit 22.

The signal delay circuit 25 sequentially applies a delay of Δt to the drive signal for driving each of the ultrasonic transducers 16, based on a delay setting signal inputted from the delay period calculating unit 27, and outputs the drive signal to the transmission/reception switching circuit 22.

### 2-7. Configuration of Reception Measuring Unit

The reception measuring unit 26 monitors the time measured by the time measuring part and measures the time taken until the ultrasonic waves are received. The reception measuring unit 26 also functions as the frequency shift amount calculating part and the reception period measuring part of the present invention.

Specifically, the reception measuring unit 26 monitors the time from a timing at which the ultrasound mode switching control unit 23 performs the process of switching to the transmission mode. Specifically, the reception measuring unit 26 monitors the time from the ultrasonic waves being transmitted from the ultrasonic arrays 12 and the time counted by the time measuring part being reset by the ultrasound mode switching control unit 23. Then, when the process of switching to the reception mode is performed by the ultrasound mode switching control unit 23, and the reception signal corresponding with the reflected ultrasonic waves received by the ultrasonic arrays 12 is inputted from the transmission/reception switching circuit 22 to the reception measuring unit 26, the time corresponding to the timing of this input (TOF data, i.e., time of flight data) is obtained, and the obtained TOF data is outputted to the central processing circuit 29.

Obtaining of the TOF data is performed according to the timing shown in FIGS. 10A and 10B. FIG. 10A is a diagram showing a model in which ultrasonic waves are transmitted from one ultrasonic array towards the blood vessel K, and FIG. 10B is a diagram showing the timing of outputting the drive signal and the timing of the reception signal.

As shown in FIG. 10A, when ultrasonic waves are transmitted from the ultrasonic array 12, a part of the ultrasonic waves is reflected at a vascular wall of the blood vessel K that is nearer the ultrasonic array (referred to as a “first vascular wall K1”), and the remainder is transmitted into the blood vessel K. When ultrasonic waves reflected at the first vascular wall K1 is received by the ultrasonic array 12, the ultrasonic array 12 outputs a reception signal Sig1.

A part of the ultrasound transmitted into the blood vessel K is reflected by blood. At this time, the wavelength shifts to correspond with the blood flow velocity. When the ultrasonic waves reflected by the blood are received by the ultrasonic array 12, the ultrasonic array 12 outputs a reception signal Sig2.

A part of the ultrasonic waves transmitted through the blood is reflected by a vascular wall that is further from the ultrasonic array 12 (referred to as a second vascular wall K2). When the ultrasonic waves reflected by the second vascular wall K2 is received by the ultrasonic array 12, the ultrasonic array 12 outputs a reception signal Sig3.

Using the timing at which the drive signal is outputted and ultrasonic waves are outputted from the ultrasonic array 12 as a reference, i.e., zero seconds, the reception measuring unit 26 measures a time T1 taken until the reception signal Sig1 is received and a time T2 taken until the reception signal Sig3 is received. Then, the reception measuring unit 26 calculates a time T3, obtained by adding the time T1 taken for the reception signal Sig1 to be received with a time equivalent to half of the time difference (i.e., (T1−T2/2) so that T3=T1+(T2−T1)/2, as the TOF, as shown in FIG. 10B.

The reception measuring unit 26 also calculates the frequency shift amount Δf, which is the difference between the frequency of the ultrasonic waves transmitted from the ultrasonic array 12 and the frequency of the ultrasonic waves received by the ultrasonic array 12, and outputs the frequency shift amount Δf to the central processing circuit 29.

### 2-8. Configuration of Delay Period Calculating Unit

The delay period calculating unit 27 calculates the drive delay period for each of the ultrasonic transducers 16 based on transmission angle data inputted from the central processing circuit 29.

The transmission angle data is data stored in the storage unit 28 in advance. Here, an example is shown in which five transmission angle data representing θs=θ1 through θ5 are stored in advance as shown in FIG. 6. A configuration in which six or more transmission angle data are stored, a configuration in which the transmission angle is varied in smaller increments, or other configurations are also possible.

Using the inputted transmission angle data θs, an element pitch d of the ultrasonic transducer 16 set in advance, and the speed of sound c, the delay period calculating unit 27 calculates the delay period Δt based on the above Equation (1), and outputs the delay period Δt as a delay setting signal to the signal delay circuit 25.

### 2-9. Configuration of Storage Unit

The storage unit 28 stores a variety of programs, data, and other information for performing the variety of processes performed by the central processing circuit 29 and the delay period calculating unit 27.

Specific examples of the variety of data include position data for the ultrasonic arrays 12 in the ultrasonic sensor 10, transmission angle data θs, TOF data, frequency shift amount data, and transmission frequency data relating to the frequency of the transmitted ultrasonic waves. Examples of the variety of programs that are recorded include a control program for controlling the entirety of the blood vessel measuring process, a reflection position calculating program for computationally obtaining the coordinate position of one point on the blood vessel K at which the ultrasonic waves have been reflected, a shift amount managing program for managing the inputted frequency shift amount data, a position calculating program for calculating the direction of blood flow (i.e., blood flow position), and a velocity calculating program for calculating the blood flow velocity.

### 2-10. Configuration of Central Processing Circuit

The central processing circuit 29 deploys the programs stored in the storage unit 28, and thereby performs a variety of processes. The central processing circuit 29 loads the shift amount managing program stored in the storage unit 28, performs processing, and thereby functions as the maximum shift amount obtaining part of the present invention. The central processing circuit 29 also loads the reflection position calculating program stored in the storage unit 28, performs processing, and thereby functions as the reflection position calculating part of the present invention. The central processing circuit 29 also loads the position calculating program stored in the storage unit 28, performs processing, and thereby functions as the movement direction measuring part of the present invention. The central processing circuit 29 also loads the velocity calculating program stored in the storage unit 28, performs processing, and thereby functions as the flow velocity calculating part of the present invention. In other words, the central processing circuit 29 forms the maximum shift amount obtaining part, the reflection position calculating part, the movement direction measuring part, and the flow velocity calculating part of the present invention.

In an instance in which, e.g., the user operates the operating part 5 to input an input signal for commencing measurement of the blood vessel position, the central processing circuit 29 outputs, to the ultrasound mode switching control unit 23, a control signal for commencing measurement.

The central processing circuit 29 also outputs, to the ultrasonic array switching circuit 21, a switching control signal for switching the ultrasonic array 12.

The central processing circuit 29 also loads the transmission angle data from the storage unit 28 and outputs the transmission angle data to the delay period calculating unit 27.

The central processing circuit 29 also performs the shift amount managing program, and thereby performs a process of obtaining the maximum frequency shift amount, which is the largest of the frequency shift amounts inputted from the reception measuring unit.

The central processing circuit 29 also executes the reflection position calculating program, and thereby performs a reflection position computation process for computationally obtaining the position at which the ultrasonic waves were reflected.

The central processing circuit 29 also executes the position calculating program, and thereby performs a blood flow direction computation process for calculating the position of the blood vessel K and ascertaining the direction of blood flow.

The central processing circuit 29 also executes the velocity calculating program, and thereby performs a blood flow velocity computation process for calculating the blood flow velocity based on the maximum frequency shift amount, the direction of blood flow, and the frequency of the transmitted ultrasonic waves.

The central processing circuit 29 also performs a process of causing the display part 4 to display the blood flow velocity and other information calculated in the variety of processes described above.

The variety of processes will be described in detail in the description for the blood flow velocity measuring method given further below.

### 3. Blood Flow Velocity Measuring Method Using Biological Testing Device

Next, a blood flow velocity measuring method using a biological testing device 1 described above will now be described with reference to accompanying drawings.

FIG. 11 is a flow chart showing a blood flow measuring process performed by the biological testing device. FIG. 12A is a diagram showing a scan area in an instance in which the transmission angle of ultrasonic waves is varied with regards to a single ultrasonic array, and FIG. 12B is a diagram showing a variation in the frequency shift amount obtained from the reception signal. FIGS. 13A to 13D are diagrams showing an example of intersecting positions of a blood vessel that intersects each of the scan areas, in relation to a blood vessel such as one shown in FIGS. 9A and 9B. FIG. 14 is a diagram showing position data with regards to the ultrasonic arrays.

In the biological testing device 1 according to the present embodiment, as described above, the ultrasonic sensor 10 is placed in close contact with the arm or another position on the body to be examined, the band 3 is tightened, and the main device body 2 is secured to the position to be examined. It thereby becomes possible to readily perform measurement of the state of the blood vessel over a long period without the need for, e.g., the user to manually hold the main device body.

Then, when the user operates the operating part 5 or otherwise inputs the input signal, the biological testing device 1 commences the blood flow velocity measuring process.

In the blood flow velocity measuring process, as shown in FIG. 11, the central processing circuit 29 of the biological testing device 1 first performs an initialization process (step S1). In this initialization process, an array variable Na and an angle variable Ns are initialized, i.e., Na and Ns are set to 1.

Next, the central processing circuit 29 performs a process of switching the ultrasonic array 12 corresponding to the array variable Na so as to be drivable (step S2). The central processing circuit 29 outputs, to the ultrasonic array switching circuit 21, a switching control signal for switching to the ultrasonic array 12A when the array variable Na is Na=1; a switching control signal for switching to the ultrasonic array 12B when the array variable Na is Na=2; a switching control signal for switching to the ultrasonic array 12C when the array variable Na is Na=3; and a switching control signal for switching to the ultrasonic array 12D when the array variable Na is Na=4.

Then, the central processing circuit 29 performs the ultrasound-transmission/reception step of the present invention. This ultrasound-transmission/reception step includes a variety of processes for the ultrasound transmission mode in step S3 and a variety of processes for the ultrasound reception mode in step S4.

In the ultrasound transmission mode, the central processing circuit 29 loads the transmission angle data θs from the storage unit 28 and outputs the transmission angle data θs to the delay period calculating unit 27. In a state in which initialization has been performed in step S1, the angle variable Ns is equal to 1. Therefore, transmission angle data θ1 is loaded and outputted to the delay period calculating unit 27. The delay period calculating unit 27 thereby calculates the delay period Δt based on Equation (1) and outputs the delay period Δt as a delay setting signal to the signal delay circuit 25.

The central processing circuit 29 also outputs, to the ultrasound mode switching control unit 23, a control signal for switching to the ultrasound transmission mode. When the control signal is inputted from the central processing circuit 29, the ultrasound mode switching control unit 23 outputs, to the transmission/reception switching circuit 22, a control signal for outputting the drive signal inputted from the signal delay circuit 25 to the ultrasonic array switching circuit 21. The ultrasound mode switching control unit 23 also provides the ultrasound signal transmission circuit 24 with an output control signal for transmitting a drive signal for driving the ultrasonic arrays 12.

A drive signal (i.e., driving pulse) to be outputted to each of the ultrasonic transducers 16 of the ultrasonic arrays 12 is outputted from the ultrasound signal transmission circuit 24 to the signal delay circuit 25. Also, in the signal delay circuit 25, the delay setting signal is inputted from the delay period calculating unit 27 as described above. Therefore, each of the drive signals is outputted to the transmission/reception switching circuit 22 with a delay whose delay period is based on the delay setting signal.

Also, the transmission/reception switching circuit 22 is switched, by the control signal inputted from the ultrasound mode switching control unit 23, to a state in which the drive signal inputted from the signal delay circuit 25 is outputted to the ultrasonic array switching circuit, as described above. Therefore, the drive signal outputted from the signal delay circuit 25, having been subjected to the delay process, is outputted via the ultrasonic array switching circuit 21 to each of the ultrasonic transducers 16 of the ultrasonic array 12 corresponding to the array variable Na.

Ultrasonic waves are thereby transmitted from the ultrasonic array 12 corresponding to the array variable Na at a transmission angle corresponding to the angle variable Ns.

The ultrasound mode switching control unit 23 also resets the time measured by the time-measuring part at the timing at which the control signal for switching to the ultrasound transmission mode is received from the central processing circuit 29 and the drive signal is outputted from the ultrasound signal transmission circuit 24, i.e., the timing at which ultrasonic waves are transmitted from the ultrasonic array 12; and measures the elapsed time. After the time taken for, e.g., one or two cycles of burst waves to be outputted has elapsed, the ultrasound mode switching control unit 23 performs the variety of processes for the ultrasound reception mode in step S4.

The ultrasound mode switching control unit 23 may also calculate, based on the delay period Δt calculated by the delay period calculating unit 27, an transmission end time at which output of the ultrasound signal from the ultrasonic transducers 16 ends, and perform a control procedure for switching to the reception mode after the transmission end time has elapsed from the timing at which ultrasonic waves are transmitted from the ultrasonic array 12.

In the ultrasound reception mode in step S4, the ultrasound mode switching control unit 23 outputs, to the transmission/reception switching circuit 22, a control signal for outputting, to the reception measuring unit 26, the reception signal inputted from the ultrasonic array switching circuit 21.

A state is thereby reached in which, when ultrasonic waves are received by the ultrasonic array 12 and the reception signal is outputted from the ultrasonic array 12, the reception signal can be inputted into the reception measuring unit 26 from the ultrasonic array switching circuit 21 via the transmission/reception switching circuit 22.

Then, in the ultrasound reception mode, the reception measuring unit 26 monitors the reception signal inputted from the transmission/reception switching circuit 22. In an instance in which the reception signal is inputted, the reception measuring unit 26 calculates the frequency shift amount and the TOF based on the reception signal (frequency-shift-amount-calculating step).

In an instance in which the blood vessel K is present in the direction of ultrasound transmission, as in the example of transmission angle θa in FIG. 12A, ultrasonic waves reflected by the vascular wall and blood is received by the ultrasonic array 12, and a reception signal is thereby outputted. In this instance, the reception measuring unit 26 calculates the time T3=T1+(T2−T1)/2 based on the timing T1, at which a reception signal Sig1 based on ultrasonic waves reflected by the first blood vessel K1 of the blood vessel K is inputted, and the timing T2, at which a reception signal Sig3 based on ultrasonic waves reflected by the second blood vessel K2 of the blood vessel K is inputted, as described above; and obtains the time T3 as the TOF data.

Also, as shown in FIGS. 10A and 10B described above, in an instance in which ultrasonic waves are reflected by the blood flowing in the blood vessel K, the frequency shifts in correspondence with the blood flow velocity. Therefore, a reception signal Sig2, which has a frequency different from that of the reception signal Sig1, is inputted into the reception measuring unit 26. Therefore, the reception measuring unit 26 calculates the frequency shift amount from the difference between the signal frequency of reception signal Sig2 and the signal frequency of the transmitted signal Sig0. In the present embodiment, the ultrasound transmission/reception process for the ultrasound transmission mode in step S3 and the ultrasound reception mode in step S4 is performed periodically based on cycle information that has been previously set. Therefore, the reception measuring unit 26 processes the ultrasound reception signal, which is received periodically, using an arithmetic algorithm that employs FFT (fast Fourier transform).

Since the reception signal Sig1 has a signal cycle that is substantially identical to that of the transmitted signal, a process for calculating the frequency shift amount from the difference between the signal frequency of the reception signal Sig1 and the signal frequency of the reception signal Sig2 may also be performed.

The reception measuring unit 26 also outputs, to the central processing circuit, the frequency shift amount data for storing the frequency shift amount, the TOF data for recording the TOF, and the reception timing data for recording the respective reception timing T1, T2 for the reception signals Sig1, Sig2. The frequency shift amount data, the TOF data, the reception timing data, and the reception data that associates the array variable Na and the angle variable Ns applicable when the above data are inputted are also stored by the central processing circuit 29 in the storage unit 28 so as to be capable of being read as appropriate.

The central processing circuit 29 subsequently adds 1 to the angle variable Ns (step S5), and judges whether or not the angle variable Ns has either reached or exceeded the maximum value NsMAX (step S6). In the present embodiment, ultrasonic waves are transmitted from an ultrasonic array at an angle that is switched between five steps; therefore, NsMAX=5. In an instance in which Ns≦NsMAX (i.e., Ns≦5 in the present embodiment) in this step S6, the central processing circuit 29 returns to the processing for the ultrasound transmission mode in step S3.

In an instance in which Ns>NsMAX (i.e., Ns>5 in the present embodiment) in step S6, the angle variable Ns is initialized and set to Ns=1, and 1 is added to the array variable Na (step S7).

The central processing circuit 29 then judges whether or not the array variable Na has exceeded the maximum value NaMAX (step S8). Since the present embodiment shows an example in which four ultrasonic arrays 12 are provided to the ultrasonic sensor 10, NaMAX is equal to 4.

In an instance in which the array variable Na is equal to or less than NaMAX (i.e., 4 in the present embodiment) in step S8, the flow returns to the process for step S2, and scanning is performed by another ultrasonic array 12. As shown in FIGS. 13A to 13D, it thereby becomes possible to scan the blood vessel K in the scan areas S1 through S4 using all of the ultrasonic arrays 12.

The central processing circuit 29 subsequently reads the shift amount managing program from the storage unit 28, executes the program, and thereby performs the maximum shift amount obtaining process (step S9: maximum-shift-amount-obtaining step).

In the maximum shift amount obtaining process, reception data is loaded from the storage unit 28, and the optimum reception data for calculating the position of the blood vessel or for measuring the blood flow is obtained.

When ultrasonic waves are transmitted from a single ultrasonic array while switching the ultrasound transmission angle, and the frequency shift amount is calculated based on the resulting reception signal, data such as that shown in FIG. 12B is obtained.

As shown in FIG. 12B, in an instance in which the blood vessel K is present within the scan area S, the frequency shift amount increases at the position at which the ultrasonic waves are reflected by the blood in the blood vessel K, and the frequency shift amount reaches the maximum value Δfa when ultrasonic waves are beamed towards the center of the blood vessel K at which the blood flow velocity is the highest (i.e., at transmission angle θa).

In the maximum shift amount obtaining process, the central processing circuit 29 first obtains the respective maximum value Δfa of the frequency shift amount for each of the ultrasonic arrays 12 from the respective frequency shift amount data obtained by transmitting ultrasonic waves from each of the ultrasonic arrays 12, where the obtained maximum value Δfa is considered an inherent frequency shift amount of each of the ultrasonic arrays 12.

The central processing circuit 29 then obtains a maximum frequency shift amount Δfmax, which is the largest of the four inherent frequency shift amounts Δfa; and a second frequency shift amount Δfnext, which has the next largest value. The central processing circuit 29 also loads reception data corresponding to the maximum frequency shift amount Δfmax and the second frequency shift amount Δfnext.

The ultrasonic array 12 and the transmission angle that corresponds to the array variable Na and the angle variable Ns associated with the maximum frequency shift amount Δfmax represent the optimum parameter for applying an appropriate ultrasound incidence angle for minimizing the error when using Equation (1) to calculate the blood flow velocity.

Next, the central processing circuit 29 reads the reflection position calculating program from the storage unit 28 and performs the reflection position computation process (step S10).

In the reflection position computation process in step S10, the central processing circuit 29 reads the TOF data, the array variable Na, and the angle variable Ns from the reception data corresponding to the maximum frequency shift amount Δfmax and the second frequency shift amount Δfnext, and calculates two points (i.e., reflection positions) in the blood vessel K. For the coordinates of the reflection position, if φ1 represents the angle of the linear scanning direction Ai of the ultrasonic array 12 corresponding to the array variable Na with respect to the x-axis (see FIG. 14), θi represents the transmission angle corresponding to the angle variable Ns, and ti represents the TOF, the coordinates of the reflection position Vi (Vxi, Vyi, Vzi) are obtained as shown in the following Equation (3). The central processing circuit 29 calculates the two reflection positions in the blood vessel K based on this Equation (3). φi represents position data showing the position of each of the ultrasonic arrays 12, stored in advance in the storage unit 28.

$$\begin{matrix}Equation ( 3 ) & \\ {V}_{i} = ( \begin{matrix}{V}_{xi} \\ {V}_{yi} \\ {V}_{zi}\end{matrix} ) = ( \begin{matrix}{X}_{i} + \frac{1}{2} {ct}_{i} \cdot sin {θ}_{i} \cdot cos {φ}_{i} \\ {Y}_{i} + \frac{1}{2} {ct}_{i} \cdot sin {θ}_{i} \cdot cos {φ}_{i} \\ \frac{1}{2} {ct}_{i} \cdot cos {θ}_{i}\end{matrix} ) & ( 3 )\end{matrix}$$

Next, the central processing circuit 29 reads the position calculating program from the storage unit 28 and performs the blood flow direction computation process (step S11: movement-direction-detecting step).

FIG. 15 is a diagram showing a model for computing the direction of blood flow. In FIG. 15, V1 is the reflection position of the blood vessel K corresponding to the maximum frequency shift amount Δfmax, and V2 is the reflection position of the blood vessel K corresponding to the second frequency shift amount Δfnext.

In the blood flow direction computation process, as shown in FIG. 15, the central processing circuit 29 calculates a vector (V1V2) (or a vector (V2V1)) from the coordinates of two reflection positions calculated in step S10, where the calculated vector is deemed to represent the direction of blood flow (i.e., the blood flow position). Specifically, in this blood vessel position measuring process, a region Sv directly below the ultrasonic sensor 10 is sufficiently small; and the blood vessel K is deemed to be positioned on a straight line linking the two reflection positions V1, V2 calculated in the above step S10. As a result, the position of the blood vessel is measured.

The central processing circuit 29 then reads the velocity calculating program from the storage unit 28, and performs the blood flow velocity computation process (step S12: flow velocity computation step).

In the blood flow velocity computation process, the central processing circuit 29 computationally obtains the flow velocity of the blood based on the Equation (1) described above. Here, the ultrasound incidence angle γ is the angle between the vector A1V1 and the vector V1V2, where A1 represents the position of the ultrasonic array 12 that detected the maximum frequency shift amount Δfmax (in the example shown in FIG. 15, the position of the ultrasonic array 12C). Therefore, the ultrasound incidence angle γ satisfies the relationship shown in the following Equation (4).

$$\begin{matrix}Equation ( 4 ) & \\ cos γ = \frac{\vec{{V}_{1} {V}_{2}} \cdot \vec{{A}_{1} {V}_{1}}}{\vert \vec{{V}_{1} {V}_{2}} \vert \vert \vec{{A}_{1} {V}_{1}} \vert} & ( 4 )\end{matrix}$$

Therefore, the above Equation (4) is substituted into Equation (1) described further above and modified, whereby the following Equation (5) is derived.

$$\begin{matrix}Equation ( 5 ) & \\ {v}_{0} = \frac{c \cdot Δ {f}_{max}}{2 {f}_{0} cos γ} = \frac{c \cdot Δ {f}_{max} \cdot \vert \vec{{V}_{1} {V}_{2}} \vert \vert \vec{{A}_{1} {V}_{1}} \vert}{2 {f}_{0} \cdot ( \vec{{V}_{1} {V}_{2}} \cdot \vec{{A}_{1} {V}_{1}} )} & ( 5 )\end{matrix}$$

The central processing circuit 29 executes the velocity calculating program, and thereby calculates blood flow velocity v0 based on the above Equation (5), using maximum frequency shift amount Δfmax, the ultrasound incidence angle γ computationally obtained from Equation (4), the speed of sound c, and the frequency f0 of ultrasonic waves transmitted from the ultrasonic arrays 12.

The biological testing device 1 periodically repeats the processes for the above steps S1 through S12, thereby making it possible to obtain the variation over time of the position of the blood vessel over a long period. In particular, the biological testing device 1 according to the present embodiment can be worn by the user, using the band 3, at all times; and performing measurement periodically as described above makes it possible to accurately ascertain the position of the blood vessel, even in an instance in which movement by the user causes a variation in the position of the blood vessel. Therefore, it becomes possible to measure the state of the blood vessel with regards to an accurate blood vessel position over a long period.

When performing this repeated process, the biological testing device 1 may designate the two ultrasonic arrays 12 that detected the maximum frequency shift amount Δfmax and the second frequency shift amount Δfnext, and repeatedly perform steps S9 through S12 based on the reception data measured by these two ultrasonic arrays. In this instance, the need to obtain the maximum frequency shift amount Δfmax and the second frequency shift amount Δfnext using the ultrasonic arrays 12 with every measurement of the blood flow velocity is obviated; it becomes possible to simplify the processing; and it becomes possible to reduce the processing load, increase the processing speed, and save energy. In contrast, repeatedly executing steps S 1 through S12 each time measurement of the blood flow velocity is periodically performed as in the present embodiment makes it possible to accurately establish the position of the blood vessel and perform measurement of the blood flow velocity even in an instance in which, e.g., the user moves actively in everyday life and the position of the blood vessel has changed, and it is therefore possible to perform measurement of the blood flow velocity to a higher degree of accuracy.

### 4. Effects of First Embodiment

As described above, in the biological testing device 1 according to the first embodiment, the ultrasonic sensor 10 has four ultrasonic arrays 12, whose respective linear scanning direction A is disposed in respectively different directions, provided on the substrate 11; and each of the ultrasonic arrays 12 is configured so as to have a linear array structure in which ultrasonic transducers 16 are arranged along the linear scanning direction. Also, the biological testing device 1 is configured such that the delay period calculated by the delay period calculating unit 27 is inputted into the signal delay circuit 25, thereby delaying the drive signal inputted into each of the ultrasonic transducers 16 of each of the ultrasonic arrays 12, and making it possible to control the compounded wave front W of the ultrasonic waves outputted from each of the ultrasonic arrays 12 in a desired direction A fan-shaped scan area can be scanned using each of the ultrasonic arrays 12. The reception measuring unit 26 of the biological testing device 1 obtains, and outputs to the central processing circuit 29, the frequency shift amount in each of the ultrasonic arrays. Also, the central processing circuit 29 reads a maximum shift amount calculating program, and thereby obtains the maximum frequency shift amount Δfmax of the frequency shift amounts.

The maximum frequency shift amount Δfmax is a frequency shift amount that corresponds with an ultrasound incidence angle γ at which computation is possible to a high degree of accuracy with minimal error when the flow velocity of the blood flow is calculated. Therefore, the biological testing device 1 obviates the need to adjust the position of the ultrasonic sensor 10, to set an appropriate ultrasound incidence angle in relation to, e.g., the orientation of the blood vessel K, or to perform other laborious tasks; and it is possible to readily obtain a frequency shift amount with respect to an appropriate ultrasound incidence angle γ.

Also, since the ultrasonic sensor 10 is configured so as to comprise a plurality of ultrasonic arrays 12 having a linear array structure, the array structure and the structure of wiring to each of the ultrasonic transducers can be made simpler, manufacturing can be more readily performed, and the manufacturing cost can be made lower when compared to, e.g., a configuration in which ultrasonic arrays having a two-dimensional array structure are arranged in a spread-out arrangement on the substrate 11.

Also, the ultrasonic array to be driven is sequentially switched, the transmission angle of the transmitted ultrasonic waves is also sequentially switched, and scanning is performed, whereby it is simple to determine the ultrasonic array 12 that has detected the maximum frequency shift amount Δfmax and the transmission angle of ultrasonic waves. Therefore, the ultrasound incidence angle γ when the maximum frequency shift amount Δfmax is detected can also be readily calculated computationally using Equation (4).

Also, in the first embodiment, steps Si through S12 are periodically repeated, whereby it is possible to measure, over a long period, the state of blood flow in the body or the variation in blood flow velocity over time. Therefore, even with regards to an abnormality in the blood flow velocity that cannot be detected using only one measurement, it is possible to discover the abnormality in the blood flow velocity using measurement performed over a long period, and it is possible to support, in a satisfactory manner, the user in maintaining health.

In the present embodiment, steps S1 through S12 are periodically repeated, and measurement of blood flow velocity is performed over a long period. However, as described above, the maximum frequency shift amount Δfmax and the second frequency shift amount Δfnext may be fixed, where only processes for steps S9 through S 14 are repeatedly performed. In this instance, although the accuracy of measurement will deteriorate if the position of the blood vessel changes, it is possible to increase the speed of processing, reduce the processing load, and save energy.

Also, in a single blood vessel measurement, transmission/reception of ultrasonic waves in the ultrasound transmission mode and the ultrasound reception mode is performed periodically according to cycle data that has been set in advance. Therefore, the reception measuring unit 26 is able to calculate the frequency displacement amount speedily and to a high degree of accuracy using an arithmetic algorithm that employs FFT, based on the reception signal, which is outputted periodically.

Also, the central processing circuit 29 executes the position calculating program, thereby loading the reception timing data associated with the maximum frequency shift amount Δfmax and the reception timing data associated with the second frequency shift amount Δfnext, and calculating the vector (V1V2). In other words, the biological testing device 1 is able to readily calculate the position of the blood vessel K computationally without aligning the linear scanning direction of an ultrasonic array 12 to the axial direction of the blood vessel K or performing other laborious tasks.

Also, by executing the velocity calculating program, the central processing circuit 29 is able to readily calculate the blood flow velocity based on Equation (5), based on the maximum frequency shift amount Δfmax, the frequency f0 of the transmitted ultrasonic waves, and the ultrasound incidence angle γ computationally obtained using Equation (4). Therefore, it is possible to obviate the need to adjust the position of the ultrasonic sensor 10 and set the ultrasound incidence angle γ to an optimum angle, or to perform other laborious tasks; and it is possible to readily calculate the flow velocity of the blood flow computationally to a high degree of accuracy.

### Second Embodiment

Next, the biological testing device according to the second embodiment of the present invention will be described with reference to the accompanying drawings. In the biological testing device 1 according to the first embodiment described above, the blood flow velocity was measured as the state of the blood vessel. However, in the biological testing device 1 according to the second embodiment, blood pressure, in addition to the blood flow velocity described above, is measured as a state of the blood vessel. In the description for the second embodiment and subsequent embodiments, configurations that are the same as those in the first embodiment are labeled with the same numerals, and corresponding descriptions are omitted or simplified.

### 1. Configuration of Biological Testing Device

The biological testing device 1 according to the second embodiment has substantially the same configuration as the first embodiment, and comprises the main device body 2 and the band 3 connected to the main device body 2 as shown in FIG. 1.

The variety of structures provided within the main device body 2 are substantially the same as the first embodiment, and are configured so as to include the ultrasonic sensor 10, the ultrasonic array switching circuit 21, the transmission/reception switching circuit 22, the ultrasound mode switching control unit 23, the ultrasound signal transmission circuit 24, the signal delay circuit 25, the reception measuring unit 26, the delay period calculating unit 27, the storage unit 28, and the central processing circuit 29.

Descriptions of configurations that are the same as those in the first embodiment are not provided in the following description.

A vascular diameter calculating program and a blood pressure calculating program for calculating the blood pressure are recorded as the variety of programs in the storage unit 28, in addition to the control program, the reflection position calculating program, the shift amount managing program, the position calculating program, and the velocity calculating program,

The central processing circuit 29 loads the vascular diameter calculating program stored in the storage unit 28, performs processing, and thereby functions as a diameter obtaining part of the present invention. The central processing circuit 29 also loads the blood pressure calculating program stored in the storage unit 28, performs processing, and thereby functions as a pressure measuring part of the present invention. In other words, the central processing circuit 29 forms the maximum shift amount obtaining part, the reflection position calculating part, the movement direction measuring part, the flow velocity calculating part, the diameter obtaining part, and the pressure measuring part of the present invention.

As with the first embodiment, in an instance in which, e.g., the user operates the operating part 5 to input an input signal for commencing measurement of the blood vessel position, the central processing circuit 29 outputs, to the ultrasound mode switching control unit 23, a control signal for commencing measurement.

The central processing circuit 29 also outputs, to the ultrasonic array switching circuit 21, a switching control signal for switching the ultrasonic array 12.

The central processing circuit 29 also loads the transmission angle data from the storage unit 28 and outputs the transmission angle data to the delay period calculating unit 27.

The central processing circuit 29 also performs the shift amount managing program, and thereby performs a process of obtaining the maximum frequency shift amount, which is the largest of the frequency shift amounts inputted from the reception measuring unit.

The central processing circuit 29 also executes the reflection position calculating program, and thereby performs a reflection position computation process for computationally obtaining the position at which the ultrasonic waves were reflected.

The central processing circuit 29 also executes the position calculating program, and thereby performs a blood flow direction computation process for calculating the position of the blood vessel K and ascertaining the direction of blood flow.

The central processing circuit 29 also executes the velocity calculating program, and thereby performs a blood flow velocity computation process for calculating the blood flow velocity based on the maximum frequency shift amount, the direction of blood flow, and the frequency of the transmitted ultrasonic waves.

In addition, the central processing circuit 29 according to the second embodiment executes the vascular diameter calculating program, and thereby performs a vascular diameter computation process for calculating the vascular diameter based on the reception timings T1, T2 of the reception signals Sig1, Sig2 inputted from the reception measuring unit 26.

The central processing circuit 29 also executes the blood pressure calculating program, and thereby performs a blood pressure computation process for calculating the blood pressure.

The central processing circuit 29 also performs a process of causing the display part 4 to display the blood flow velocity, the blood pressure, and other information calculated in the variety of processes described above.

### 2. Blood Pressure Measuring Method Using Biological Testing Device

Next, a blood pressure measuring method using the biological testing device 1 described above will be described with reference to the accompanying drawings. FIG. 16 is a flow chart showing a blood pressure measurement process using the biological testing device.

As shown in FIG. 16, in the biological testing device 1 according to the second embodiment, the blood flow velocity v0 is calculated using the same method as that used in the first embodiment, i.e., by performing steps S1 through S12.

According to the blood pressure measurement of the second embodiment, in the maximum shift amount obtaining process in step S9, a minimum frequency shift amount Δfmin, which has the smallest value of the four inherent frequency shift amounts Δfa, and a third frequency shift amount Δfnmin_next, which has the next smallest value, are obtained in addition to the maximum frequency shift amount Δfmax, which is the largest, and the second frequency shift amount Δfnext, which has the next largest value.

As shown in FIG. 16, after the blood flow velocity computation process in step S12, the central processing circuit 29 loads the vascular diameter calculating program from the storage unit 28, and performs a vascular diameter calculating program (step S13; flow-path-diameter obtaining step). Here, the central processing circuit 29 loads the reception timing data (reception timing T1, T2) from the reception data corresponding to the minimum frequency shift amount Δfmin obtained in step S9, and calculates the vascular diameter based on the corresponding time difference (T2−T1) and the speed of sound c.

In the vascular diameter calculating program, the central processing circuit 29 may also obtain, as the vascular diameter, an average value of a diameter calculated from the reception timing data associated with the minimum frequency shift amount Δfmin and a diameter calculated from the reception timing data associated with the third frequency shift amount Δfnmin_next. The central processing circuit 29 may also calculate the diameter from each of the reception timing data associated with the four inherent frequency shift amounts Δfa, and use the average value of the diameters as the vascular diameter.

Then, the central processing circuit 29 reads the blood pressure calculating program from the storage unit 28 and performs the blood pressure computation process (step S14: blood-pressure-measuring step).

In the blood pressure computation process, the central processing circuit 29 computationally calculates the blood pressure based on the flow velocity v0 of the blood flow calculated in step S12 and the vascular diameter D calculated in step S13.

FIGS. 17A and 17B show drawings illustrating the flow of blood in the blood vessel K. FIG. 17A is a schematic diagram showing an enlargement of a part of the blood vessel K, and FIG. 17B is a diagram showing a distribution of velocity of blood in the blood vessel K.

As shown in FIG. 17A, a model is posited in which an r-axis extends in a radial direction that is orthogonal to the axis of the blood vessel, using the axis of the blood vessel as the center axis (i.e., the x-axis). Blood flowing in the blood vessel K is a laminar flow in a vein, and is also a laminar flow in an artery in regions near the periphery. In a state of laminar flow of such description, blood flowing in the blood vessel K follows the relationship according to the following general Equation (6).

$$\begin{matrix}Equation ( 6 ) & \\ \frac{d}{d r} ( r \frac{d u}{d r} ) = \frac{r}{μ} \frac{d P}{d x} & ( 6 )\end{matrix}$$

In the above Equation (6), μ represents the viscosity coefficient of the fluid, P represents the pressure of the fluid, and u represents the velocity of the fluid. At the boundary portion between the vascular wall and the blood, the flow velocity of the blood flow is zero; therefore, the following Equation (7) is derived from the above Equation (6).

$$\begin{matrix}Equation ( 7 ) & \\ u = \frac{1}{4 μ} ( - \frac{d P}{d x} ) ( {d}^{2} - {r}^{2} ) & ( 7 )\end{matrix}$$

The above Equation (7) shows that the flow velocity of blood is at its maximum along the center axis of the blood vessel K (i.e., the x-axis), and the flow velocity of blood is zero at the boundary portion with respect to the vascular wall, as shown in FIG. 17B. Integrating the velocity u around the center axis of the blood vessel K (i.e., the x-axis) makes it possible to obtain the flow rate Q of the blood passing through the cross-section of the blood vessel K, and the following Equation (8) can be derived.

$$\begin{matrix}Equation ( 8 ) & \\ Q = {\int }_{0}^{d} u \cdot 2 π r d r = \frac{π {d}^{4}}{8 μ} ( - \frac{d P}{d x} ) & ( 8 )\end{matrix}$$

Since the blood flow velocity is at its maximum at the center axis of the blood vessel K (i.e., the x-axis; r=0), the maximum velocity u0 can be represented by the following Equation (9).

$$\begin{matrix}Equation ( 9 ) & \\ {u}_{0} = \frac{{d}^{2}}{4 μ} \cdot ( - \frac{d P}{d x} ) & ( 9 )\end{matrix}$$

Therefore, from the above Equations (8) and (9), it becomes possible to obtain the flow rate Q from the vascular diameter D and the maximum flow velocity u0 as shown in the following Equation (10).

$$\begin{matrix}Equation ( 10 ) & \\ Q = \frac{π {D}^{2} \cdot {u}_{0}}{8} & ( 10 )\end{matrix}$$

(dP/dx) in Equation (9) represents an equation indicating the pressure gradient of the blood vessel K with respect to the center axis direction. If, for example, the respective pressure at both end parts of the blood vessel K within a range having a length L is P1 and P2, the relationship shown in the following Equation (11) is satisfied.

$$\begin{matrix}Equation ( 11 ) & \\ \frac{d P}{d x} = \frac{{P}_{2} - {P}_{1}}{L} & ( 11 )\end{matrix}$$

By substituting Equation (11) into Equation (8) described further above, the following Equation (12) is derived.

$$\begin{matrix}Equation ( 12 ) & \\ Q = \frac{( {P}_{1} - {P}_{2} )}{\frac{8 μ L}{π {d}^{4}}} & ( 12 )\end{matrix}$$

The blood flow resistance R is defined as shown in the following Equation (13).

$$\begin{matrix}Equation ( 13 ) & \\ R = \frac{μ C}{{d}^{4}} \\ Where C is a constant & ( 13 )\end{matrix}$$

Using this Equation (13) makes it possible to replace Equation (12) with the equation QR=P1−P2. Also, in peripheral blood vessels, P2 can be regarded as zero; therefore, blood pressure P at a predetermined blood vessel position can be regarded as equal to pressure P1. Therefore, the following Equation (14) is satisfied.

Equation (14)

P=QR (14)

The central processing circuit 29 can use the above Equations (10), (13), and (14) to calculate the blood pressure. Specifically, the central processing circuit 29 uses the flow velocity v0 of the blood flow calculated in step S12 and the vascular diameter D calculated in step S13, and calculates the flow rate Q using the above Equation (10). Then, the central processing circuit 29 uses the vascular diameter D calculated in step S13 and calculates the blood flow resistance R using the above Equation (13). The central processing circuit 29 then calculates the blood pressure P based on Equation (14), using the flow rate Q and the blood flow resistance R.

As with the first embodiment, the biological testing device 1 periodically repeats the process for steps S1 through S14 described above, thereby making it possible to obtain the variation over time of the position of the blood vessel over a long period. In particular, the biological testing device 1 according to the present embodiment can be worn by the user, using the band 3, at all times; and performing measurement periodically as described above makes it possible to accurately ascertain the position of the blood vessel, even in an instance in which movement by the user causes a variation in the position of the blood vessel. Therefore, it becomes possible to measure the state of the blood vessel (i.e., blood flow, blood pressure, pulse, etc.) with regards to an accurate blood vessel position over a long period.

As with the first embodiment, when performing this repeated process, the biological testing device 1 may designate the two ultrasonic arrays 12 that detected the maximum frequency shift amount Δfmax and the second frequency shift amount Δfnext, and repeatedly perform steps S9 through S14 based on the reception data measured by these two ultrasonic arrays. In this instance, the need to obtain the maximum frequency shift amount Δfmax and the second frequency shift amount Δfnext using the ultrasonic arrays 12 with every measurement of the blood pressure is obviated; it becomes possible to simplify the processing; and it becomes possible to reduce the processing load, increase the processing speed, and save energy. In contrast, repeatedly executing steps S1 through S 14 each time measurement of the blood pressure is periodically performed as in the present embodiment makes it possible to accurately establish the position of the blood vessel and perform measurement of the blood pressure even in an instance in which, e.g., the user moves actively in everyday life and the position of the blood vessel has changed, and it is therefore possible to perform measurement of the blood pressure to a higher degree of accuracy.

### 3. Effects of Second Embodiment

In the second embodiment described above, the following effects can be attained in addition to the effects of the first embodiment described above.

Specifically, the central processing circuit 29 is able to calculate the vascular diameter D based on the reception timing data using the vascular diameter calculating program, and to readily calculate the blood pressure P from the vascular diameter D and the flow velocity v0 of the blood flow determined from Equation (5) using Equations (10), (13), and (14).

Therefore, it is possible to obviate the need to adjust the position of the ultrasonic sensor 10, set the ultrasound incidence angle γ to an optimum angle, or perform other laborious tasks; and it is possible to readily obtain a blood pressure computationally to a high degree of accuracy.

Also, in the second embodiment, as with the second embodiment, steps S1 through S14 are periodically repeated, whereby it is possible to measure, over a long period, the state of blood flow in the body or the variation in blood flow velocity and the blood pressure over time. Therefore, even with regards to an abnormality in the blood flow velocity or the blood pressure that cannot be detected using only one measurement, it is possible to discover the abnormality using measurement performed over a long period, and it is possible to support, in a satisfactory manner, the user in maintaining health.

### Third Embodiment

Next, a biological testing device that is a measuring device according to the third embodiment of the present invention will now be described with reference to the accompanying drawings.

FIG. 18 is a top view showing the plane of the substrate of an ultrasonic sensor 10A of the biological testing device according to the third embodiment of the present invention.

In the biological testing device 1 according to the first embodiment and the second embodiment described above, reception data based on the reception signal inputted from the ultrasonic sensor 10 is used to obtain the vector V1V2, perform measurement of the position of the blood vessel, and computationally obtain the vascular diameter D. In contrast, in the biological testing device 1 according to the third embodiment, the ultrasonic sensor 10A has position-measuring ultrasonic arrays 17 for measuring the position of the blood vessel and the vascular diameter, arranged on outer peripheral sides of the ultrasonic arrays 12. In other words, the position-measuring ultrasonic arrays 17 also function as the diameter-measuring ultrasonic arrays of the present invention.

Each of the position-measuring ultrasonic arrays 17 is arranged at a substantially central part of each of the sides of the substrate 11. Each of the position-measuring ultrasonic arrays 17 is configured by arranging a plurality of ultrasonic transducers 16 along a linear scanning direction B. The linear scanning direction B of each of the position-measuring ultrasonic arrays 17 is disposed in the same direction as the linear direction of the respective side on which the corresponding position-measuring ultrasonic array 17 is arranged. In the present embodiment, since the substrate 11 is formed so as to have a square shape, each of the position-measuring ultrasonic arrays 17E, 17G has a linear scanning direction B that is disposed along the direction of the x-axis, and each of the position-measuring ultrasonic arrays 17F, 17H has a linear scanning direction B that is disposed along the direction of the y-axis, as shown in FIG. 18.

The position-measuring ultrasonic arrays 17 have a configuration that is substantially the same as the ultrasonic arrays 12. Specifically, the substrate 11 is provided with opening parts 111 for forming the diaphragms 141 of the ultrasonic transducers 16 forming each of the ultrasonic arrays 12, and is also provided, at the central part of each of the sides, with opening parts 111 for forming the diaphragms 141 of the ultrasonic transducers 16 forming each of the position-measuring ultrasonic arrays 17. The opening parts 111 of each of the position-measuring ultrasonic arrays 17 and the opening parts 111 of each of the ultrasonic arrays 12 have configurations in which, e.g., the respective opening diameters are different and the frequencies that can be transmitted are different. Specifically, in the ultrasonic arrays 12, the opening parts 111 are formed so as to be capable of outputting ultrasonic waves of e.g., 2 Hz, and so as to be capable of outputting a frequency at which the amount of frequency shift due to the Doppler effect can be readily obtained. In contrast, in the position-measuring ultrasonic arrays 17, the opening parts 111 are formed so as to be capable of outputting ultrasonic waves of, e.g., 10 Hz, and so as to be capable of outputting a frequency at which the position of the blood vessel or the vascular diameter can be readily detected due to a short ultrasound wavelength.

FIG. 19 is a diagram showing a scan area of a single position-measuring ultrasonic array 17.

In each of the position-measuring ultrasonic arrays 17, as with the ultrasonic arrays 12, the transmission angle of ultrasonic waves can be controlled by a drive signal, to which a delay has already been applied, outputted from the signal delay circuit 25. As shown in FIG. 19, each of the position-measuring ultrasonic array 17 thereby has a fan-shaped scan area Sarea that is coplanar with respect to a plane that is disposed along the linear scanning direction B and orthogonal to the plane of the substrate 11. In the second embodiment, the position-measuring ultrasonic arrays 17 of such description are provided with respect to each of the sides, and the scan area Sarea is formed so as to cover each of the sides. In other words, each of the side surfaces of the region directly below the substrate 11 is surrounded by the scan area Sarea of each of the position-measuring ultrasonic arrays 17. In an ultrasonic sensor 10A having scan areas Sarea of such description, in an instance in which the blood vessel K passes through the region directly below the substrate 11, the blood vessel K intersects at least two of the scan areas Sarea. Therefore, detecting the intersection point using ultrasound makes it possible to computationally obtain the position of the blood vessel.

In a biological testing device 1 according to the second embodiment of such description, the position calculating program, the velocity calculating program, and the vascular diameter calculating program stored in the storage unit 28 cause the central processing circuit 29 to perform processes that are different to those in the first embodiment. The processes performed by the central processing circuit 29 as a result of these programs will now be described.

The central processing circuit 29 performs, using the position calculating program, a blood flow direction computation process for computationally obtaining the position of the blood vessel, based on the reception data outputted from the position-measuring ultrasonic arrays 17.

Specifically, in the blood flow direction computation process according to the second embodiment, the central processing circuit 29 loads the reception data outputted from the position-measuring ultrasonic arrays 17, and obtains the corresponding TOF data. Then, based on the TOF data, the central processing circuit 29 calculates two points on the blood vessel K, i.e., two points at which the ultrasonic waves transmitted from the position-measuring ultrasonic arrays 17 has been reflected, and calculates a straight line linking the two points as a provisional position of the blood vessel.

Also, in the reflection position computation process, the central processing circuit 29 calculates the reflection positions V1,V2 based on the TOF data associated with the maximum frequency shift amount Δfmax, and judges whether or not the reflection positions V1, V2 are positioned on the provisional position of the blood vessel. If it is judged that the reflection positions V1, V2 are positioned on the provisional position of the blood vessel, the central processing circuit 29 obtains the provisional position of the blood vessel as the position of the blood vessel. In an instance in which the reflection positions V1, V2 are not positioned on the provisional position of the blood vessel, a correction is performed so that a line passing through the two reflection positions detected by the position-measuring ultrasonic arrays 17 and the points V1, V2 is deemed the position of the blood vessel. In this instance, the axial direction of the blood vessel K is a curved line, rather than a straight line.

The central processing circuit 29 also performs, using the velocity calculating program, the blood flow velocity computation process. In an instance in which it was judged in the blood flow direction computation process that the reflection positions V1, V2 are positioned on the provisional position of the blood vessel, the same process as that in the first embodiment described above is performed.

In an instance in which it was judged in the blood flow direction computation process that the reflection positions V1, V2 are not positioned on the provisional position of the blood vessel, the direction of the tangent of the blood vessel K in the axial direction at the reflection position V1 is judged as the direction of blood flow, and the angle γ between the direction of the tangent and the vector A1V1 is calculated. The flow velocity v0 of the blood flow is then calculated based on Equation (5) in the same manner as in the first and the second embodiments.

Then, the central processing circuit 29, with regards to the vascular diameter calculating program, loads the reception data based on the reception signal from the position-measuring ultrasonic arrays 17 and calculates the vascular diameter D from the reception timing data.

### Effects of Third Embodiment

In the biological testing device according to the third embodiment described above, the ultrasonic sensor 10A comprises the position-measuring ultrasonic arrays 17, and measurement of the position of the blood vessel is performed based on the reception signal outputted from the position-measuring ultrasonic arrays 17.

In an instance in which ultrasonic waves of a single frequency is transmitted from the ultrasonic arrays 12, and both the frequency shift amount for measuring the blood flow velocity and the TOF data or reception timing data for measuring the position of the blood vessel and the vascular diameter are obtained, since the ultrasound reflectivity of the vascular wall or the blood differs, error is likely to be present in one of the data. In contrast, in the third embodiment, the position-measuring ultrasonic arrays 17 are provided, thereby making it possible to transmit ultrasonic waves having a frequency for measuring the position of the blood vessel or the vascular diameter and ultrasonic waves having a frequency for measuring the frequency shift amount from different elements. Therefore, it is possible to measure a more accurate position of the blood vessel and vascular diameter using the reception signal outputted from the position-measuring ultrasonic arrays 17, and it is possible to measure a more accurate frequency shift amount using the reception signal outputted from the ultrasonic arrays 12. It is thereby possible to calculate a more accurate blood flow velocity and blood pressure, and to increase the accuracy of measurement.

### Fourth Embodiment

Next, the biological testing device according to the fourth embodiment of the present invention will now be described with reference to the accompanying drawings.

The biological testing device according to the fourth embodiment is one in which the structure of the ultrasonic arrays 12 of the biological testing device 1 according to the first embodiment and the second embodiment has been modified. Other configurations are the same as those in the biological testing device 1 according to the first embodiment and the second embodiment.

FIG. 20 is a top view of a substrate 11 of an ultrasonic sensor 10B of the biological testing device according to the fourth embodiment.

As with the first embodiment, the ultrasonic sensor 10B of the biological testing device according to the fourth embodiment has ultrasonic arrays 31 (31A, 31B, 31C, 31D) arranged at the central part of the substrate 11.

As with the first embodiment, each of the ultrasonic arrays 31 has a linear scanning direction A that is disposed in respectively different directions, and a plurality of ultrasonic elements 32 are arranged along the linear scanning direction A. Each of the ultrasonic elements 32 comprises a plurality (three, in the example of the present embodiment) of ultrasonic transducers 16, arranged along an orthogonal-to-scan direction, which is orthogonal to the linear scanning direction A. The configuration of each of the ultrasonic transducers 16 is the same as that of the ultrasonic transducers 16 according to the first embodiment, and a description will be omitted from the following description.

In a single ultrasonic element 32, e.g., an upper electrode wire 153A of each of the ultrasonic transducers 16 is connected to each other as a common electrode, and a lower electrode wire 151A connected to each of the lower electrodes 151 of each of the ultrasonic transducers 16 is provided independently of each other. In other words, the ultrasonic transducers 16 are configured so as to be capable of being driven independently of each other.

A configuration is also possible in which all of the upper electrode wires 153A are connected to form a common electrode wire in a single ultrasonic array 31.

In each of the ultrasonic arrays 31 of such description, it is possible to control the ultrasound output timing for each of the ultrasonic transducers 16 in each of the ultrasonic elements 32 to focus the ultrasonic waves onto a single predetermined point. It is thereby possible to expand the Fresnel zone within which a planar ultrasonic wave can be outputted, and to extend the distance over which ultrasonic waves are propagated as planar waves from the ultrasonic transducers 16.

FIG. 21 is a diagram showing the difference between the Fresnel zone of ultrasonic waves outputted from a single ultrasonic transducer (top) and the Fresnel zone of an ultrasonic array according to the second embodiment (bottom).

As shown in the top drawing in FIG. 21, with ultrasonic waves transmitted from a singular ultrasonic transducer 16 as shown in the first embodiment described above, the range up to a distance of L1=D2/4λ, represents the Fresnel zone, and planar waves are propagated. In contrast, as shown in FIG. 12B, the timing at which ultrasonic waves are transmitted from the ultrasonic element 32 at the center is delayed with respect to the transmission timing of ultrasonic waves transmitted from the ultrasonic elements 32 at both end parts, whereby compounded waves compounded from ultrasonic waves transmitted from each of the ultrasonic transducers 16 are formed so as to focus on a focus point P that corresponds with the delay period. In other words, controlling the ultrasound transmission timing for each of the ultrasonic transducers 16 makes it possible to control the position of the focus point P and to adjust the distance L2 representing the Fresnel zone (i.e., the distance over which the ultrasonic waves are propagated as planar waves) to a desired distance.

This controlling of the delay period can be performed using the central processing circuit 29, the delay period calculating unit 27, and the signal delay circuit 25. A method of calculating this delay period will now be described with reference to FIG. 22.

FIG. 22 is a diagram showing a state in which ultrasonic waves transmitted from a plurality of ultrasonic transducers 16 is focused at a predetermined single point. In an instance in which 1 to N ultrasonic transducers 16 are arranged and the ultrasound transmission timing at each of the ultrasonic transducers 16 is adjusted to focus the ultrasonic waves to a focus point P as in the example shown in FIG. 22, the time τ(i, F) taken for ultrasonic waves to reach the focus point P from an ultrasonic transducer 16 located at an arbitrary point Yi can be represented using the following Equation (15).

$$\begin{matrix}Equation ( 15 ) & \\ τ ( i , F ) = \frac{\sqrt{{( {y}_{1} - F tan θ )}^{2} + F}}{c} & ( 15 )\end{matrix}$$

This Equation (15) is an arithmetic equation representing an instance in which the center point of the driven roller 32 represents the origin (0,0), the ultrasonic transducers 16 are arranged along a y-axis, and ultrasonic waves are transmitted in the direction of the x-axis, as shown in FIG. 22. In the above Equation (5), F represents the x-coordinate position of the focus point, and θ represents the angle between the x-axis and a straight line passing through the center point of the driven roller 32 (i.e., the origin) and the focus point P.

In the biological testing device according to the fourth embodiment, the central processing circuit 29, the delay period calculating unit 27, and the signal delay circuit 25 controls the output timing of the drive signal applied to each of the ultrasonic transducers 16 based on the above Equation (15), and delays the transmission timing of ultrasonic waves.

Specifically, in the biological testing device according to the fourth embodiment, the central processing circuit 29 sets, according to the depth at which the blood vessel K is positioned, a focus point P onto which the ultrasonic waves are to be focused, and outputs the focus point P to the delay period calculating unit 27. Based on the above Equation (15), the delay period calculating unit 27 thereby calculates the delay period for focusing the ultrasonic waves outputted from each of the ultrasonic transducers 16 onto the focus point P, and inputs the delay period into the signal delay circuit 25.

The depth at which the blood vessel K is positioned may be one that is inputted by, e.g., the user operating the operating part 5. For example, in an instance in which the portion to be examined is a finger or another portion at which the distance between the skin and the blood vessel K is small, when the user operates the operating part 5 to input an input signal to indicate that the object of examination is a finger, the central processing circuit 29 outputs, to the delay period calculating unit 27, a value for F (i.e., the distance to the focus point P) that has a small value. Also, in an instance in which, e.g., the portion to be examined is an arm or another portion at which the distance from the skin to the blood vessel K is larger, when the user operates the operating part 5 to input an input signal to indicate that the object of examination is an arm, the central processing circuit 29 outputs, to the delay period calculating unit 27, a value for F that has a larger value. Also, in an instance in which, e.g., the portion to be examined is a leg or another portion at which the distance from the skin to the blood vessel K is even larger, when the user operates the operating part 5 to input an input signal to indicate that the object of examination is a leg, the central processing circuit 29 outputs, to the delay period calculating unit 27, a value for F that has an even larger value. A value that has been set in advance and stored in the storage unit 28 can be used as the value for F. A configuration is also possible in which, in an instance in which the portion to be examined is, e.g., a finger, no delay is applied to the transmission timing of ultrasonic waves between each of the ultrasonic transducers 16 forming a single ultrasonic element 32, and the position of the blood vessel is detected using the Fresnel zone as with the first embodiment described further above.

The ultrasonic sensor 10B may again also be configured so that position-measuring ultrasonic arrays 17 as described in the third embodiment are separately provided. In such an instance, the position-measuring ultrasonic arrays 17 may also be configured so as to have an array structure in which a plurality of ultrasonic transducers 16 are arranged in a direction that is orthogonal to the linear scanning direction B, and in which it is possible to expand the Fresnel zone and extend the distance over which ultrasonic waves are propagated as planar waves, thereby increasing the range within which it is possible to measure the position of the blood vessel K.

### Effects of Fourth Embodiment

In the biological testing device according to the fourth embodiment described above, each of the ultrasonic arrays 31 comprises ultrasonic elements 32 having a linear array structure, arranged along the linear scanning direction A; and each of the ultrasonic elements 32 comprises ultrasonic transducers 16 arranged in the orthogonal-to-scan direction, which is orthogonal to the linear scanning direction A. Based on the optimum value for F inputted from the central processing circuit 29, the delay period calculating unit 27 and the signal delay circuit 25 outputs, to each of the ultrasonic transducers 16 arranged in the linear scanning direction of each of the ultrasonic elements 32, a drive signal for progressively delaying the timing of ultrasound transmission from the ultrasonic transducers 16 at both end parts to the ultrasonic transducer 16 at the central part.

Therefore, it is possible to increase the distance L2 representing the Fresnel zone of ultrasonic waves transmitted from each of the ultrasonic transducers 16 (i.e., the distance over which ultrasonic waves are propagated as planar waves) compared to an instance in which a singular ultrasonic transducer 16 is used, and to increase the size of the scan areas Sarea within which the position of the blood vessel can be scanned. Therefore, it is possible to increase the size of the region within which the biological testing device is able to perform measurement of the position of the blood vessel K.

### Fifth Embodiment

Next, a biological testing device according to the fifth embodiment of the present invention will now be described with reference to the accompanying drawings.

The biological testing device according to the fifth embodiment is one in which the structure of the ultrasonic arrays 12 of the biological testing device 1 according to the first embodiment and the second embodiment has been modified. Other configurations are the same as those in the biological testing device 1 according to the first embodiment and the second embodiment.

FIG. 23 is a top view of an ultrasonic sensor 10C of the biological testing device according to the fifth embodiment.

In the biological testing device according to the fifth embodiment, eight ultrasonic arrays 12, whose respective linear scanning directions A are respectively different, are arranged on the substrate 11 of the ultrasonic sensor 10C. Specifically, the ultrasonic array 12A has a linear scanning direction A1 that is parallel to the x-axis, and comprises a plurality of ultrasonic transducers 16 arranged along the linear scanning direction A1. The ultrasonic array 12B has a linear scanning direction A2 that is parallel to the y-axis, and comprises a plurality of ultrasonic transducers 16 arranged along the linear scanning direction A2. The ultrasonic array 12C has a linear scanning direction A3 that is inclined at an angle of 45° with respect to the x-axis, and comprises a plurality of ultrasonic transducers 16 arranged along the linear scanning direction A3. The ultrasonic array 12D has a linear scanning direction A4 that is inclined at an angle of 135° with respect to the x-axis, and comprises a plurality of ultrasonic transducers 16 arranged along the linear scanning direction A4. The ultrasonic array 12E has a linear scanning direction A5 that is inclined at an angle of 30° with respect to the x-axis, and comprises a plurality of ultrasonic transducers 16 arranged along the linear scanning direction A5. The ultrasonic array 12F has a linear scanning direction A6 that is inclined at an angle of 60° with respect to the x-axis, and comprises a plurality of ultrasonic transducers 16 arranged along the linear scanning direction A6. The ultrasonic array 12G has a linear scanning direction A7 that is inclined at an angle of 120° with respect to the x-axis, and comprises a plurality of ultrasonic transducers 16 arranged along the linear scanning direction A7. The ultrasonic array 12H has a linear scanning direction A8 that is inclined at an angle of 135° with respect to the x-axis, and comprises a plurality of ultrasonic transducers 16 arranged along the linear scanning direction A8.

In a biological testing device comprising the ultrasonic sensor 10C of such description, it is possible to obtain the maximum frequency shift amount Δfmax based on the frequency shift amount outputted from the eight ultrasonic arrays 12. Therefore, it is possible to obtain, from a large amount of data, data that is more suitable for measurement of the blood flow, and to computationally determine a blood flow velocity to a higher degree of accuracy with minimal error.

### Sixth Embodiment

Next, a biological examination system 1A according to a sixth embodiment of the present invention will now be described with reference to the accompanying drawings. FIG. 24 is a perspective view showing an overview of a biological examination system 1A according to the sixth embodiment of the present invention.

In FIG. 24, the biological examination system 1A according to the sixth embodiment comprises an ultrasonic sensor 10D, a band 3, and a control device 7. The ultrasonic sensor 10D and the control device 7 form the measuring device according to the present invention. FIG. 25 is a block diagram showing an overview of a configuration of the biological examination system according to the sixth embodiment.

In the biological examination system 1A, the ultrasonic sensor 10D formed into a film shape is secured using the band to a predetermined portion on the body to be examined, and a signal outputted from the ultrasonic sensor 10D is processed using the control device 7. Although not shown in FIG. 24, an acoustic matching part 61 is formed on a front layer of the ultrasonic sensor 10D.

Also, as shown in FIG. 25, an ultrasonic array switching circuit 21, a transmission/reception switching circuit 22, an ultrasound mode switching control unit 23, an ultrasound signal transmission circuit 24, a signal delay circuit 25, a reception measuring unit 26, a delay period calculating unit 27, a storage unit 28, a central processing circuit 29, and other components are built into the control device 7.

Each of the configurations provided to the control device 7 is the same as according to the first embodiment described further above, and a description will be omitted from the following description.

A configuration is possible in which an operating part 5, using which the user operates the biological examination system 1A, and a display part 4 are provided to the control device 7. A configuration is also possible in which the operating part 5 and the display part 4 are connected to the control device 7.

FIG. 24 shows an example in which the control device 7 is connected to the control device 7 by a wired connection. However, a configuration is also possible in which, e.g., an infrared light, Bluetooth®, radio wave, or another wireless connection is established. In such an instance, a wireless communication part for performing wireless communication is provided to each of the ultrasonic sensor 10D and the control device 7. In an instance in which a configuration of such description is present, it is possible to provide a biological examination system 1A that is superior in terms of portability, in which no wire presents an obstruction when the ultrasonic sensor 10D is being worn.

In a biological examination system IA according to the fifth embodiment of the above description, only a thin-type ultrasonic sensor 10D is secured to the body. Therefore, even in an instance in which the state of the blood vessel is examined over a long period, the biological examination system 1A does not present an obstruction or present a burden on the body due to weight. Therefore, e.g., the user is able to measure the variation in the state of the blood vessel while continuing normal life.

### Other Embodiments

The present invention is not limited to the embodiments described above, and includes modifications, improvements, and other embellishments of such a scope that the object of the present invention can be achieved.

For example, in the first through sixth embodiments described above, the biological testing device 1 and the biological examination system 1A for measuring the flow velocity or the blood pressure of blood flowing in the blood vessel K in the body are shown as examples. However, these are not provided by way of limitation. For example, the present invention can be applied for any measuring device for measuring the flow velocity or the pressure of a measured fluid flowing in a tube, in an instance in which the tube is provided within an accommodating body that is capable of transmitting ultrasonic waves and the tube has a different acoustic impedance to the accommodating body. For example, the present invention can be applied to a measuring device such as one for measuring the position of pipework arranged in a liquid or the flow velocity or the pressure of a measured fluid within the pipework.

Also, in the first through sixth embodiments described above, an example was shown in which each of the ultrasonic transducers 16 is configured by laminating a film-shaped lower electrode 151, piezoelectric film 152, and upper electrode 153 on a support film 14. However, these are not provided by way of limitation; it being also possible to have a configuration in which, e.g., a piezoelectric body that is bulky (i.e., shaped as a mass of indeterminate form) is arranged on the support film 14. In such an instance, the bulky piezoelectric body is formed into a rectangular shape that can be readily formed by cutting, the longitudinal direction of the rectangle is disposed along the orthogonal-to-scan direction, and a plurality of piezoelectric bodies are arranged along the linear scanning direction A.

Also, in the first through sixth embodiments described above, for the ultrasonic transducers 16, examples were shown for a configuration in which the shape of the diaphragm 141 is circular, and a circular piezoelectric body 15 is formed. However, these are not provided by way of limitation. A configuration is also possible in which a piezoelectric body 15 having, e.g., a rectangular or a polygonal shape is provided to a diaphragm 141 having a rectangular or a polygonal shape. Specifically, it is possible to freely design the shape of the ultrasonic transducers 16, taking the balance of stress during vibration of the diaphragm 141 and other factors into account.

Also, examples were shown in which a single ultrasonic transducer 16 is used to perform both transmission and reception of ultrasonic waves; however, a configuration is also possible in which transducers for ultrasound transmission and transducers for ultrasound reception are provided as separate elements.

Also, in the first through sixth embodiments, an example was shown in which the substrate 11 of the ultrasonic sensor 10 is formed so as to be rectangular (i.e., square). However, these are not provided by way of limitation; the substrate 11 may be formed as another polygon, a circle, an ellipse, or any other shape.

In the third embodiment, in an instance in which the substrate 11 is polygonal, each of the position-measuring ultrasonic arrays 17 is arranged with respect to each of the sides; and in an instance in which the substrate 11 is circular or elliptical, each of the position-measuring ultrasonic arrays 17 is arranged so that the linear scanning direction B follows the direction of a tangent of the substrate 11, whereby it is possible to measure the position of the blood vessel K passing through the region directly below the ultrasonic sensor 10A.

In the first through sixth embodiments, in the reflection position computation process in step S10, the central processing circuit 29 calculates the reflection position V2 from reception data corresponding to the second frequency shift amount Δfnext. However, the reflection position V2 may be calculated from reception data corresponding to another inherent frequency shift amounts Δfa.

In the fourth embodiment, an example was shown for a configuration where the position at which the biological testing device 1 is worn is set and inputted, whereby the central processing circuit 29 selects the optimum value for F and outputs the value to the delay period calculating unit 27, and the delay period calculating unit 27 calculates a delay period with respect to a drive signal outputted to each of the ultrasonic transducers 16 forming each of the ultrasonic elements 32; however, these are not provided by way of limitation. For example, in a biological testing device 1 that is exclusively for use at a position at which the object of examination of the state of the blood vessel is set in advance, e.g., for examination of the state of the blood vessel in the arm, a configuration is also possible in which the delay period calculating unit 27 calculates the delay period based on an optimum value for F that has been set in advance.

Also, in the first through sixth embodiments, an example is shown in which a single ultrasonic array 12 is arranged with respect to a single linear scanning direction A. However, a configuration is also possible in which a plurality of ultrasonic arrays 12 are arranged with respect to a single linear scanning direction A.

Also, in the sixth embodiment, an example of a configuration was shown in which the ultrasonic array switching circuit 21, the transmission/reception switching circuit 22, the ultrasound mode switching control unit 23, the ultrasound signal transmission circuit 24, the signal delay circuit 25, the reception measuring unit 26, the delay period calculating unit 27, the storage unit 28, and the central processing circuit 29 are included in the control device 7. However, these are not provided by way of limitation. For example, a configuration is also possible in which the ultrasonic array switching circuit 21, the transmission/reception switching circuit 22, the signal delay circuit 25, and other circuits are provided to the ultrasonic sensor 10D. In an instance in which each of the circuits is configured in the ultrasonic sensor 10D, forming the circuits on, e.g., the substrate 11 makes it possible to minimize any increase in the thickness dimension of the ultrasonic sensor 10D.

Also, in the first through sixth embodiments described above, an example was shown in which, in each of the ultrasonic arrays 12, each of the ultrasonic transducers 16 performs both the transmission and reception of ultrasonic waves, and the ultrasound mode switching control unit 23 performs switching between the ultrasound transmission mode and the ultrasound reception mode. However, these are not provided by way of limitation.

For example, of the ultrasonic transducers 16 forming each of the ultrasonic arrays 12, each of the ultrasonic transducers 16 arranged at an odd-numbered position may be used as an element for ultrasound transmission, and each of the ultrasonic transducers 16 arranged at an even-numbered position may be used as an element for ultrasound reception. A configuration is also possible in which ultrasonic waves are transmitted from ultrasonic transducers 16 located on the side towards one end of the line in each of the ultrasonic arrays 12, and ultrasonic waves are received by ultrasonic transducers 16 located on the side towards the other end of the line.

A configuration is also possible in which ultrasonic transducers exclusively for ultrasound transmission and ultrasonic transducers exclusively for ultrasound reception are provided as separate structures.

In such an instance, a configuration is also possible in which exclusively ultrasound-transmitting arrays 121, in which a plurality of exclusively ultrasound-transmitting transducers 161 are arranged in a straight line; and exclusively ultrasound-receiving arrays 122, in which a plurality of exclusively ultrasound-receiving transducers 162 are arranged in a straight line, are provided parallel to each other as shown in FIG. 26.

Also, in a modified measurement system such as one described above or the biological examination system 1A according to the fifth embodiment, the control device 7 may be configured so as to transmit data to a server device connected via, e.g., an Internet connection. In such an instance, it is possible to monitor, at all times, the state of the blood vessel of a patient wearing an ultrasonic sensor 10D in, e.g., a hospital or another medical facility.

In the second embodiment, an example is shown in which, during the blood flow direction computation process, in an instance in which the reflection position V1, which has been computationally obtained based on TOF data associated with the maximum frequency shift amount Δfmax, is not present on the position of the blood vessel computationally obtained from the reflection position coordinates based on the reception signal from the position-measuring ultrasonic arrays 17, the central processing circuit 29 corrects the position of the blood vessel using reflection position coordinates based on the reception signal from the position-measuring ultrasonic arrays 17 and reflection positions V1, V2 based on the reception signal from the ultrasonic arrays 12. However, these are not provided by way of limitation. For example, the position of the blood vessel may be computationally obtained solely from reception data based on the reception signal from the position-measuring ultrasonic arrays 17, where a correction is performed in which the V1 coordinates is moved onto the position of the blood vessel.

Also, in the embodiments described above, an example was shown in which the delay period calculating unit 27 is a device for receiving transmission angle data from the central processing circuit 29 and thereby computationally obtains the delay period for the drive signal inputted into each of the ultrasonic transducers 16, i.e., an example in which the delay period calculating unit 27 is configured as hardware. However, these are not provided by way of limitation. For example, a configuration is also possible in which a delay period calculating program is stored in the storage unit 28, and the delay period calculating program is ready and executed by the central processing circuit 29, whereby the delay period is computationally obtained for each of the drive signals.

Also, an example was shown in which the central processing circuit 29 reads and executes the shift amount managing program, the reflection position calculating program, the position calculating program, the velocity calculating program, the vascular diameter calculating program, and the blood pressure calculating program, and thereby functions as the maximum shift amount obtaining part, the reflection position calculating part, the movement direction calculating part, the flow velocity calculating part, the diameter obtaining part, and the pressure measuring part of the present invention. However, the maximum shift amount obtaining part, the reflection position calculating part, the movement direction calculating part, the flow velocity calculating part, the diameter obtaining part, and the pressure measuring part may also be configured as hardware from, e.g., an integrated circuit.

Although specific descriptions were given above concerning preferred aspects for carrying out the present invention, these are not provided by way of limitation to the present invention. Specifically, although the present invention has been expressly illustrated and described mainly in relation to specific embodiments, those skilled in the art may perform a variety of modifications and/or improvements on the embodiments described above without departing from the technical concepts and scope of the object of the present invention.

### General Interpretation of Terms

In understanding the scope of the present invention, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the turns, “including”, “having” and their derivatives. Also, the terms “part,” “section,” “portion,” “member” or “element” when used in the singular can have the dual meaning of a single part or a plurality of parts. Finally, terms of degree such as “substantially”, “about” and “approximately” as used herein mean a reasonable amount of deviation of the modified term such that the end result is not significantly changed. For example, these terms can be construed as including a deviation of at least ±5% of the modified term if this deviation would not negate the meaning of the word it modifies.

While only selected embodiments have been chosen to illustrate the present invention, it will be apparent to those skilled in the art from this disclosure that various changes and modifications can be made herein without departing from the scope of the invention as defined in the appended claims. Furthermore, the foregoing descriptions of the embodiments according to the present invention are provided for illustration only, and not for the purpose of limiting the invention as defined by the appended claims and their equivalents.

What is claimed is:

1. A measuring device comprising:
an ultrasonic sensor having a substrate with a plurality of ultrasonic arrays each having a linear array structure in which a plurality of ultrasonic elements are arranged along a linear scanning direction so that linear scanning directions along which the ultrasonic elements are arranged are different from each other at least for two of the ultrasonic arrays that are arranged on the substrate;
a transmission/reception control unit configured to control the ultrasonic arrays to transmit/receive ultrasonic waves; and
a computation part configured to measure a frequency shift amount based on ultrasonic waves received by the ultrasonic arrays.

2. The measuring device according to claim 1, wherein
the transmission/reception control unit includes a delay control unit configured to control a transmission angle of ultrasonic waves transmitted from the ultrasonic arrays.

3. The measuring device according to claim 1, wherein
the computation part includes
a frequency shift amount calculating part configured to calculate, for each of the ultrasonic arrays, the frequency shift amount based on a reception signal outputted from each of the ultrasonic arrays, the frequency shift amount being a difference between a frequency of transmitted ultrasonic waves and a frequency of received ultrasonic waves, and
a maximum shift amount obtaining part configured to obtain a maximum frequency shift amount, which is the largest of the frequency shift amounts calculated for each of the ultrasonic arrays by the frequency shift amount calculating part.

4. The measuring device according to claim 3, wherein
the transmission/reception control unit is configured to control the ultrasonic sensor to transmit/receive ultrasonic waves at a plurality of timings, and
the frequency shift amount calculating part is configured to calculate the frequency shift amount at each of the timings, based on the reception signal outputted from one of the ultrasonic arrays corresponding to the maximum frequency shift amount obtained in the maximum shift amount obtaining part at a previous timing.

5. The measuring device according to claim 3, wherein
the transmission/reception control unit is configured to control the ultrasonic sensor to transmit/receive ultrasonic waves at a plurality of timings,
the frequency shift amount calculating part is configured to calculate the frequency shift amount at each of the timings based on the reception signal outputted from each of the ultrasonic arrays, and
the maximum shift amount obtaining part is configured to obtain the maximum frequency shift amount from the frequency shift amount each time a calculation is made by the frequency shift amount calculating part.

6. The measuring device according to claim 4, wherein
the transmission/reception control unit is configured to control the ultrasonic sensor to periodically transmit/receive ultrasonic waves.

7. The measuring device according to claim 3, wherein
the computation part includes
a reception period measuring part configured to measure a reception period between transmission of ultrasonic waves and reception of reflected ultrasonic waves in the one of the ultrasonic arrays corresponding to the maximum frequency shift amount or another one of the ultrasonic arrays,
a reflection position calculating part configured to calculate a reflection position at which ultrasonic waves are reflected, based on data relating to a position of the ultrasonic array, the reception period, and a transmission angle at which ultrasonic waves are transmitted from the ultrasonic array, and
a movement direction measuring part configured to determine a direction of movement of a measured fluid from the reflection position calculated by the reflection position calculating part.

8. The measuring device according to claim 3, wherein
the ultrasonic sensor includes a plurality of position-measuring ultrasonic arrays configured and arranged to measure a position of a tube through which a measured fluid flows, and
the computation part includes a movement direction calculating part configured to calculate the direction of movement of the measured fluid in the tube based on a reception signal outputted from the position-measuring ultrasonic arrays.

9. The measuring device according to claim 7, further comprising
a flow velocity calculating part configured to calculate a flow velocity of the measured fluid based on the direction of movement of the measured fluid, the maximum frequency shift amount, and a frequency of ultrasonic waves transmitted from the ultrasonic array.

10. The measuring device according to claim 9, further comprising
a diameter obtaining part configured to obtain a diameter of a flow path in which the measured fluid flows, and
a pressure measuring part configured to measure a pressure of the measured fluid based on the diameter of the flow path and the flow velocity of the measured fluid.

11. The measuring device according to claim 10, wherein
the ultrasonic sensor includes a plurality of diameter-measuring ultrasonic arrays configured and arranged to measure the diameter of the flow path, and
the diameter obtaining part is configured to calculate the diameter of the flow path based on a reception signal outputted from the diameter-measuring ultrasonic arrays.

12. A biological testing device comprising:
the measuring device according to claim 1, and
an acoustic matching part covering a surface of the ultrasonic arrays in the ultrasonic sensor, the acoustic matching part having an acoustic impedance that is equivalent to an acoustic impedance of a living body.

13. A biological testing device comprising:
the measuring device according to claim 2, and
an acoustic matching part covering a surface of the ultrasonic arrays in the ultrasonic sensor, the acoustic matching part having an acoustic impedance that is equivalent to an acoustic impedance of a living body.

14. A biological testing device comprising:
the measuring device according to claim 3, and
an acoustic matching part covering a surface of the ultrasonic arrays in the ultrasonic sensor, the acoustic matching part having an acoustic impedance that is equivalent to an acoustic impedance of a living body.

15. A biological testing device comprising:
the measuring device according to claim 4, and
an acoustic matching part covering a surface of the ultrasonic arrays in the ultrasonic sensor, the acoustic matching part having an acoustic impedance that is equivalent to an acoustic impedance of a living body.

16. A biological testing device comprising:
the measuring device according to claim 5, and
an acoustic matching part covering a surface of the ultrasonic arrays in the ultrasonic sensor, the acoustic matching part having an acoustic impedance that is equivalent to an acoustic impedance of a living body.

17. A biological testing device comprising:
the measuring device according to claim 6, and
an acoustic matching part covering a surface of the ultrasonic arrays in the ultrasonic sensor, the acoustic matching part having an acoustic impedance that is equivalent to an acoustic impedance of a living body.

18. A biological testing device comprising:
the measuring device according to claim 7, and
an acoustic matching part covering a surface of the ultrasonic arrays in the ultrasonic sensor, the acoustic matching part having an acoustic impedance that is equivalent to an acoustic impedance of a living body.

19. A flow velocity measuring method for measuring a flow velocity of a measured fluid using an ultrasonic sensor having a plurality of ultrasonic arrays arranged on a substrate, each of the ultrasonic arrays having a linear array structure in which a plurality of ultrasonic elements are arranged along a linear scanning direction to transmit/receive ultrasonic waves so that linear scanning directions are different from each other for the ultrasonic arrays, the flow velocity measuring method comprising:
controlling a transmission angle at which ultrasonic waves are transmitted from each of the ultrasonic arrays and performing transmission of ultrasonic waves from the ultrasonic arrays and reception of reflected ultrasonic waves;
calculating, for each of the ultrasonic arrays, a frequency shift amount based on a reception signal outputted from each of the ultrasonic arrays, the frequency shift amount being a difference between a frequency of transmitted ultrasonic waves and a frequency of received ultrasonic waves;
obtaining a maximum frequency shift amount, which is the largest of the frequency shift amounts calculated for each of the ultrasonic arrays in the calculating of the frequency shift amount; and
calculating the flow velocity of the measured fluid based on the frequency of transmitted ultrasonic waves, the maximum frequency shift amount, and a direction of movement of the measured fluid.

20. A method for measuring a pressure of a measured fluid comprising:
measuring the flow velocity of the measured fluid by the flow velocity measuring method of claim 19;
obtaining a flow path diameter of the measured fluid; and
calculating the pressure of the measured fluid based on the flow path diameter and the flow velocity of the measured fluid.
